(19) United States
(12) Patent Application Publication
(10) Pub. No.: US 2026/0033818 A1
(43) Pub. Date: Feb. 5, 2026

(54) DEVICE AND METHOD FOR CLOSING AN OPENING OF A BODY WALL

(71) Applicant: KATHOLIEKE UNIVERSITEIT LEUVEN K.U. LEUVEN RESEARCH & DEVELOPMENT, Leuven (BE)

(72) Inventors: Allan JAVAUX, Leuven (BE); Luc JOYEUX, Leuven (BE); Jean-Jacques BONDONNEAU, Leuven (BE); Jeroen MEYBOSCH, Leuven (BE)

(21) Appl. No.: 18/992,624

(22) PCT Filed: Jul. 12, 2023
(86) PCT No.: PCT/EP2023/069369

§ 371 Date: Jan. 9, 2025

(30) Foreign Application Priority Data
Jul. 12, 2022 (EP) 22184322.0

(51) Int. Cl. A61B 17/00

(52) U.S. Cl. CPC A61B 17/0057; A61B 2017/00004; A61B 2017/00477; A61B 2017/00579; A61B 2017/00588; A61B 2017/0061; A61B 2017/00623; A61B 2017/00659

(57) ABSTRACT

A biomedical device (100) for closing an opening (1261) of a body wall (1260), comprising: a central component (101), and a plurality of arms (120) movably connected to the central component; wherein the arms (120) comprise an elongated arm portion (121) and penetration means (122) comprising a barb (123); wherein the biomedical device is configured such that: a) the elongated arm portions are oriented in a first direction when the device is in a delivery configuration; b) the elongated arm portions are oriented in a plane perpendicular to the first direction, and the penetration means are located at a first distance (R1) from the central component, when the device is in a deployed configuration; and c) the elongated arm portions are oriented in said plane, and the penetration means are located at a second distance (R2) from the central component, smaller than the first distance (R1), when the device is in a retracted configuration.

### FIELD OF THE INVENTION

The present invention relates in general to the field of biomedical devices, and more in particular to implantable biomedical devices and methods for closing an opening during or after a minimally invasive surgery (MIS). The present invention also relates to a method of assembling such a device.

### BACKGROUND OF THE INVENTION

Minimally Invasive Surgery (MIS) is a surgical procedure less invasive than open surgery used for similar indications. It decreases patient's morbidity and allows patients to have less pain and to return to their daily activities more quickly. MIS includes many surgical procedures: endoscopic techniques, laparoscopy (digestive, urologic and gynecological surgery), thoracoscopy, mediastinoscopy, cervicoscopy, robotic surgery and fetoscopy (maternal-fetal surgery). MIS also concerns similar surgical specialties in veterinary surgery. MIS is carried out through the wall of a body cavity after the placement of a trocar or through an anatomical opening.

MIS typically involves use of an endoscope and long instruments inserted inside the body through trocars. These instruments are manipulated by the surgeon to perform an operation with indirect observation of the surgical field through an endoscope displayed on a large-scale video screen.

For all MIS procedure, the trocar opening needs to be closed at the end of the procedure to avoid post-operative complications, such as trocar site hernia, loss of body fluids or infections. The closure remains difficult due to tissue wall thickness preventing efficient internal closure.

Various devices and techniques to close a trocar opening following an MIS procedure have been proposed in the art.

WO2016083606(A1) discloses a biomedical device for watertight sealing of a port opening after a minimally invasive surgery (MIS). The device comprises arms and flaps, which arms can be folded open, and which flaps can be brought into an edge-to-edge arrangement.

EP1836968(A1) discloses a biomedical device for sealing a vessel puncture or closing a cardiac septal defect. The device comprises a tubular member and a plurality of struts.

US2010106171(A1) discloses a medical device for creating an anastomosis between the inferior vena cava (IVC) and the mesenteric portal vein. It comprises a proximal flange part, a distal flange part, and a flow lumen between them. The anastomosis device is made of a memory shape material.

WO2017075467(A1) discloses a fascial wound closure device comprising an applicator releasably attached to an implant main body. The implant main body has a plurality of connecting members configured to extend from the main body. A lattice is formed between the connecting members. In each connecting member, an engagement hook is mounted to close a fascial wound by engaging into body tissue. While such membrane may provide a sealing of the opening, e.g. a watertight sealing, the present invention may be primarily concerned with reducing the size of, or (preferably) substantially closing, an opening in a tissue wall, e.g. an interior tissue wall, without necessarily providing a tight seal on the wound, e.g. to enable faster healing and/or reducing the risk of causing a hernia. Nonetheless, embodiments of the present invention may advantageously provide a sufficient closure (and/or sealing) of the opening without requiring such membrane. It will be understood that the use of a membrane may also have various disadvantages, such as added complexity, costs and/or risks, such as a of misfolding or constriction.

### SUMMARY OF THE INVENTION

It is an object of embodiments of the present invention to provide a biomedical device for closing (or at least reducing the size of) an opening from the body wall of a human or an animal, e.g. during or after a minimally invasive surgery (MIS). The opening may have been created for performing said MIS.

It is also an object of embodiments of the present invention to provide a method of assembling such a biomedical device.

It is also an object of embodiments of the present invention to provide a kit of parts for building or assembling such a biomedical device.

It is also an object of embodiments of the present invention to provide a method of closing (or at least reducing the size of) an opening from the body wall of a human or an animal using such a biomedical device, e.g. during or after a minimally invasive surgery (MIS).

It is also an object of embodiments of the present invention to provide a biomedical device that allows closure (or at least approaching) of the inner layers of the abdominal wall therefore potentially improving (e.g. faster) healing of the tissue surrounding the opening and/or preventing complications of MIS.

It is also an object of embodiments of the present invention to provide a biomedical device that is easier to assemble, and/or that consists of fewer parts.

These and other objectives are accomplished by a biomedical device, a method of assembling, a kit of parts, and a method of closing an opening according to embodiments of the present invention.

According to a first aspect, the present invention provides a biomedical device for closing an opening, or at least reducing the size of an opening of a human or animal body wall, e.g. during or after a minimally invasive surgery (MIS), the device comprising: a central component (e.g. a base) having a central axis (also referred to as “reference axis”, e.g. A); and a plurality of arms (e.g. at least two, at least three, at least four, at least five, at least six arms) movably connected (directly or indirectly) to the central component. The plurality of arms are movably, e.g. hingably, connected to the central component so as to allow the device to transform between different configurations, e.g. by movement (e.g. rotation and/or sliding) of the arms. The different configurations of the device comprise at least a delivery configuration, a deployed configuration and a retracted configuration. The arms comprise an elongated arm portion, and penetration means for penetrating said body wall. The biomedical device is configured such that: a) the elongated arm portions form an angle (e.g. @) smaller than 20° relative to the central axis when the device is in a delivery configuration; and b) the elongated arm portions are oriented substantially perpendicular to the central axis, and a tip of the penetration means is located at a first distance (e.g. R1) from the central axis when the device is in a deployed configuration; and c) the elongated arm portions are oriented substantially perpendicular to the central axis, and the tip of the penetration means is located at a second distance (e.g. R2) from the central axis, smaller than the first distance (e.g. R1), when the device is in a retracted configuration. The penetration means comprises a barb for anchoring the penetration means in said body wall, e.g. such as to resist release of the barbed penetration means once it is engaged in the tissue.

In preferred embodiments, the biomedical device is implantable in a human or animal body. The “central axis” is an axis passing through a central location of the central component. The central axis is preferably an axis of rotational symmetry. It is an advantage of this biomedical device that it facilitates an opening of a body (e.g. an opening in a wall of a body) to be closed from the inside by requiring only manipulations by the surgeon from the outside. This allows to close the deepest (innermost) structural tissue layer of the abdominal wall, in turn reducing the risk of strangulation/herniation of bowel into the entry site (port site herniation). The arms may be connected to the central component, directly or indirectly, by means of “connection means”. The connection means are preferably located eccentric. The connection means may comprise for example hinges, e.g. living hinges, or clamping means.

The act of moving the arms from an orientation substantially parallel to the central axis into an orientation substantially orthogonal to the central axis may also be referred to herein as “spreading the arms” or “rotating the arms”.

The biomedical device may further comprise: means for retracting said arms, e.g. threads or sutures.

In some embodiments, the penetration means are located near the ends of the arms. In this case, the elongated arm portion and the penetration means may together form a L-shape.

In some embodiments, the penetration means are located at a non-zero distance from the ends of the arms. In this case, the elongated arm portion and the penetration means may together form a T-shape.

In some embodiments, the penetration means and elongated arm portions are curved, e.g. such that the penetration means can move in a continuous motion relative to the elongated arm portion, e.g. by sliding in and out of the elongated arm portion along said curve. Thus, in this case, the elongated arm portion and the penetration means may together form an (e.g. cornerless) arc or C-shape.

In an embodiment, the biomedical device further comprises a plurality of guiding elements hingedly connected to the central component (e.g. base), wherein the guiding elements are configured for receiving respective elongated arm portions; and wherein each arm is slidingly connected to a respective guiding element.

While not absolute required, the guiding elements may have a tubular shape, and the arms may have a shape fitting inside this tubular shape. The guiding elements may have an outer surface with rounded edges, for preventing inadvertently cutting into the wall. In the context of the present invention, the expression “hingedly connected” may mean that two parts are connected to each other, but are capable of rotating relative to one another. Examples of such connection means may be described as follows: (i) “cooperating or collaborative connection means” including a male and female part, the male part comprising a protrusion, the female part comprising a cavity, the protrusion fitting in the cavity, wherein the male and female part are capable to rotate relative to one another, or as (ii) “cooperating or collaborative connection means” comprising a plurality of first parts having a through opening and second parts having a through opening and holding means, e.g. a thread or a suture or a pin passing through said through-openings for holding the first and second parts together while allowing rotation of the second parts relative to the first parts.

Preferably the guiding elements and the arms are provided with a clicking mechanism that allow the arms to be engaged with the guiding elements (e.g. inside or partially inside the guiding elements) during assembly, and that prevent the arms from being disengaged.

In an embodiment, the guiding elements have a shape configured for contacting each other in an edge-to-edge arrangement so as to form a relatively smooth surface, when the device is in the deployed and the retracted configuration. Thus, the guiding elements may be shaped so as to fit together when abutting on each other in the deployed and the retracted configuration, so as to jointly present (e.g; form) a smooth surface, e.g. a continuous surface, e.g. without surface irregularities, discontinuities, ridges, edges (e.g. except the joint periphery/boundary) or the like. Or, said otherwise, the relevant surfaces presented by the individual guiding elements (in said configuration) may be shaped so as to align to their neighboring surfaces (e.g. by substantially coinciding edges). Particularly, the surface jointly formed by the guiding elements (in deployed and retracted configuration) may refer to a surface oriented toward, or oriented away from, the body tissue wall (in use of the device), e.g. a jointly formed surface orthogonal to (or substantially or approximately orthogonal to) the axial direction (again, in the deployed and/or retracted configuration).

Examples of such an embodiment are shown in FIG. 22D to FIG. 22G.

In an embodiment, the central component (e.g. base) further comprises a plurality of clamping means, configured for receiving a respective arm portion between them; and wherein the arms further comprise an elongated slot or a trench or a groove for engaging with the clamping means.

It is an advantage of such clamping means that the arm is rotatable from the first direction to the second direction when the arm is engaged at a first position of said slot or trench or groove (e.g. as illustrated in FIG. 21A), and that the arm is slidable with respect to the base when the arm is oriented in the second direction (e.g. as illustrated in FIG. 21B and FIG. 21C), for allowing the penetration means (e.g. pin or hook) to move from said first distance (R1) to said second distance (R2) from the centre of the base.

In an embodiment, the clamping means comprise pairs of pillars extending from the base, and a protruding (e.g. cylindrical) element extending from each pillar for engaging or clamping the arms between them. An example of such an arrangement is shown in FIG. 21D.

In an embodiment, the clamping means comprise pairs of pillars extending from the base, and an element, e.g. a cylindrical element, interconnecting these pillars. An arm with an elongated slot can be situated between the pillars, and the cylindrical element may extend through said slot. An example of such an arrangement is shown in FIG. 21E.

In an embodiment, the penetration means have an overall tapering shape or an overall pointed shape (e.g. have a pin shape or a hook shape). The penetration means furthermore comprises said barb, e.g. distinct from the end (tip) of said tapering/pointed shape.

Preferably the penetration means have a sharp tip or a sharp end. Preferably this overall pointed shape is oriented substantially perpendicular to the elongated arm portion. An example of such an arm with such penetration means is shown in FIG. 1E and FIG. 24A. The shape and/or size of the penetration means can vary depending on the application, e.g. for abdominal surgery, thoracic surgery, maternal-fetal surgery, etc.
It is noted that the term “barb” is well known, e.g. as a part of a typical fishing hook. The barb may comprise a second pointed shape, smaller than the overall pointed shape, and pointing in a direction substantially opposite to the direction of the overall pointed shape. It is advantage of using penetration means with a barb that it allows the biomedical device to be anchored in the wall, and that the risk of involuntarily disengaging is dramatically reduced.

In an embodiment, the central component is a base having a first side with means for connecting the arms (directly, or indirectly, e.g. by means of guiding elements), and having a second side with a smooth surface. Again, a smooth surface may generally refer to a continuous surface, e.g. without surface irregularities, discontinuities, ridges or the like.

The smooth surface may be substantially planar or substantially dome shape. It is an advantage that the first side of the base has a smooth surface. This reduces the risk of potential injuries caused by organs coming into contact with the base. In this embodiment, the guiding elements and/or the arms ay rest on the first side of the base, when the biomedical device is in the deployed or retracted configuration. Thus the base helps to limit the guiding elements and/or the arms from rotating over an angle larger than about 90° with respect to their initial orientation (which they had when the device was in the delivery configuration).

In an embodiment, the central component has a rotational symmetrical shape about the central axis (A) of an order in the range from 2 to 8.

In an embodiment, a projection of the central component is a substantially circular shape, or a substantially elliptical shape, or a substantially polygonal shape (e.g. square, hexagonal, octagonal).

In an embodiment, a projection of the biomedical device, when in its deployed configuration and its retracted configuration, has a rotational symmetrical shape of an order in the range from 2 to 8.

In preferred embodiments, the biomedical device has a rotational symmetry of order 3 (360°/3=120°), or of order 4 (360°/4=90°), or of order 5 (360°/5=72°), or of order 6 (360°/6=60°), or of order 8 (360°/8=45°).

In an embodiment, the biomedical device is configured such that the elongated arm portions are substantially radially oriented when the device is in the deployed configuration and in the retracted configuration.

The arms may be angularly spaced by a constant angle equal to 360° divided by the number of arms.

In an embodiment, the biomedical device is made of a biocompatible material.

In an embodiment, the biomedical device is made of a bioabsorbable material.

The biomedical device may comprise for example one or more of the following materials: polydioxanone (PDS) or polyglactine 910. Polydioxanone (PDS) is commercially available from Ethicon under the brand Absolok®. Another bioabsorbable material is polyglycolic acid (PGA) or polylactic acid (PLA), or self-reinforcing (SR), or a bioabsorbable copolymer of PLA, PGA and/or SR. But the present invention is not limited hereto, and other bioabsorbable materials can also be used.

In an embodiment, the central component (e.g. a base) has an interface for allowing a temporal connection to an external device (e.g. a driver) for allowing positioning and/or reconfiguration of the biomedical device.

The external device may be a so called “driver”, and the “interface” or “external connection means” may comprise a cavity or a blind hole for receiving an end of a tube which is selectively connectable to and releasable from the central component, e.g. by means of first threads or sutures (e.g. as illustrated in FIG. 14E).

In an embodiment, the central component (e.g. the base) may comprise a circular opening with an inner groove, and the external tube may be a substantially cylindrical tube with movable parts (e.g. two spherical balls) which may be selectively retracted inside the tube, or radially extending from the tube for engaging with the inner groove of the base.

In an embodiment, the central component (e.g. the base) may comprise a circular groove for receiving an end of an external tube, and the central component may further comprise two through-openings (e.g. inside the circle defined by said groove) for allowing passage of a thread or suture for connecting the external tube to the central component (e.g. when the device is in the delivery configuration), and for disconnecting the external tube from the central component (e.g. when the device is in the retracted configuration).

In an embodiment, the base further comprises a further opening for passing an endoscope, or a passageway made of a see-through material for connecting an endoscope.

In an embodiment, the central component is integrally formed with the plurality of guiding elements, which are connected to the central component by means of living hinges. The arms can be attached thereto during an assembly stage.

In an embodiment, the biomedical device has an external diameter in the range from 3 mm to 30 mm in the delivery configuration, or in the range from 5 mm to 25.0 mm, or in the range from 7.0 mm to 18.0 mm, e.g. equal to about 3 mm, or equal to about 4 mm, or equal to about 5 mm, or equal to about 7 mm, or equal to about 10 mm, or equal to about 12 mm, or equal to about 15 mm, or equal to about 18 mm, or equal to about 20 mm, or equal to about 25 mm, or equal to about 30 mm.

According to a second aspect, the present invention also provides a method of assembling a biomedical device according to the first aspect; the method comprising the steps of: a) providing said central component (e.g. a base); b) providing said at least two elongated arms, and connecting them (directly or indirectly) to the central component via said connection means.

In an embodiment, step a) may comprise: providing a base, and providing a plurality of guiding elements, and connecting the guiding elements to the base.

In an embodiment, step a) may comprise: providing a central component integrally formed with a plurality of guiding elements connected thereby by means of living hinges.

In an embodiment wherein the central component is a base, and wherein the connection means comprise guiding elements hingedly connected to the base, step b) may comprise: providing at least two guiding elements, and connecting said guiding elements to the base, and connecting the arms to the guiding elements.

In an embodiment wherein the central component is integrally formed with a plurality of guiding elements by means of living hinges, step b) may comprise: connecting the arms to the guiding elements.

In an embodiment, the method further comprises one or more of the following steps: providing a first tube (also referred to herein as “inner tube”), and releasably connecting the first tube and the central component (e.g. the base), e.g. by means of a thread or suture; providing a second tube (also referred to herein as “outer tube”), and rotating the arms so as to substantially align them in the first direction (e.g. axial direction, substantially parallel to the first tube), and inserting at least the arms and optionally also the central component (e.g. the base) inside the second tube.

The method may further comprise the step of: attaching one or more strings or sutures to at least some, or all elongated arms, or looping one or more strings or sutures through openings of said at least some, or all elongated arms. These strings or sutures may be guided through the above mentioned tube(s), and preferably have a length longer than that of the tube(s) such that a portion extends beyond an end of the tube, for allowing an operator, e.g. a surgeon, to pull on said strings or sutures from outside the body, to thereby provide the necessary force for the elongated arms to slide towards each other (e.g. radially inwards), thereby bringing the biomedical device in the retracted configuration.

According to the third aspect, the present invention also provides a kit of parts for building a biomedical device according to the first aspect, the kit of parts comprising: said central component (e.g. base); and a plurality of arms comprising said penetration means for penetrating the wall of said body. The kit of parts may further comprise: a plurality of guides.

According to the fourth aspect, the present invention also provides a method of closing (or at least reducing the size of an opening of a human or animal body wall using a biomedical device according to the first aspect, the method comprising the steps of: a) providing the biomedical device in its delivery configuration; b) inserting the biomedical device in the opening; c) rotating the arms (and the guiding elements, if present) of the biomedical device relative to the central component, thereby bringing the biomedical device in its deployed configuration; d) moving the biomedical device towards the opening, such that the penetration means penetrates tissue surrounding the opening; e) moving at least some of the arms towards each other, thereby bringing the biomedical device in the retracted state, and reducing or closing the opening.

Step a) may further comprise: connecting a driver to the biomedical device. The driver may comprise an inner tube and an outer tube.

Step b) may comprise inserting the outer tube with the biomedical device in the opening.

Step c) may comprise shaking the biomedical device and/or rotating the biomedical device around its longitudinal axis to bring the arms out of the first orientation (e.g. substantially parallel to the tube), e.g. by making use of centrifugal forces. This step may be omitted if the arms automatically open up once they have moved outside the second tube.

Step c) may comprise moving a tubular element, e.g. a spring or the outer tube against the arms, for pushing the arms into said plane, e.g. so as to form an angle of about 90° with the central axis of the central component.

Step c) may comprise moving the outer tube to allow the biomedical device to move to the deployment configuration automatically, e.g. like a memory-shaped object would retrieve its unconstrained position. This may be achieved e.g. by connecting the arms to the central component using living hinges, which are biased outwardly, especially when the arms are forced into the second tube (during assembly), to form an angle smaller than about 20° or about 15° or about 10° with the central axis.

Step d) may comprise: removing the outer tube from the opening (in the body wall), before or after moving the central component (and thus the entire biomedical device) towards the opening.

Step e) may comprise: moving said arms towards each other, e.g. by moving the arms radially inwards, by pulling one or more threads or sutures that are connected to or looped through the arms, and that are guided through the tube(s) of the driver to a space outside the body where the surgeon can manipulate them.

The method may further comprise a step f) of disconnecting the driver from the biomedical device, e.g. by cutting threads or sutures that are guided through an inner tube of the driver and form a loop around or through the central component. By cutting these threads or sutures, the loop is broken, and the central component is disconnected from the driver.

The method may comprise a further a step g) of making (on the outside of the body wall) a knot with the threads or sutures that are connected to, or looped through the arms, and/or placing a clip on these threads or sutures in order to maintain the biomedical device in the retracted configuration. It is noted that making such a knot, or mounting such a clip is not absolutely required, especially in case the penetration means have barbs preventing them from disengaging from the body wall, and in case the arms and the guiding element have a clicking mechanism or a snap-fit mechanism or the like, preventing the arms from moving out of the guiding element after being retracted.

In an embodiment, the method further comprises a step h) of stitching the tissue on the outside of the body, e.g. to stitch the skin together.

According to another aspect, the present invention also provides an assembly comprising: a biomedical device according to the first aspect, configured in the delivery configuration; and a driver comprising a first tube and a second tube, the first tube being connected to the biomedical device by means of at least one thread or suture, and the second tube surrounding the first tube and surrounding the biomedical device.

This assembly may further comprise at least one dissolvable thread or suture connected to at least one of the arms, and guided through the first tube.

Particular and preferred aspects of the invention are set out in the accompanying independent and dependent claims. Features from the dependent claims may be combined with features of the independent claims and with features of other dependent claims as appropriate and not merely as explicitly set out in the claims.

These and other aspects of the invention will be apparent from and elucidated with reference to the embodiments described hereinafter.

### BRIEF DESCRIPTION OF THE DRAWINGS

FIG. 1A shows (from left to right) a side view of an arm, a guiding element, and a base as may be used to form a biomedical device proposed by the present invention.

FIG. 1B shows an example of a biomedical device proposed by the present invention, comprising a base, six guiding elements and six arms. The device is shown in its deployed configuration.

FIG. 1C shows a cross-sectional view, and FIG. 1D shows a perspective view of the guiding element of FIG. 1A.

FIG. 1E shows a side view of the arm of FIG. 1A.

FIG. 1F shows an example of a subassembly comprising an arm engaged with a guiding element.

FIG. 2A to FIG. 7B show different configurations of the biomedical device mounted to a driver.

FIG. 2A shows a perspective view, and FIG. 2B shows a side view of the biomedical device of FIG. 1B, configured in its “delivery configuration”, and connected to an illustrative “driver”.

FIG. 2C to FIG. 2G illustrate in more detail how a biomedical device proposed by the present invention, e.g. the biomedical device of FIG. 1B, can be connected to such a driver, to form an assembly.

FIG. 2C shows the biomedical device of FIG. 1B. in delivery configuration.

FIG. 2D shows a so called “inner tube” of the driver. In the example, the inner tube basically consists of two hollow, cylindrical portions, connected together, one portion having a small diameter, one portion having a large diameter. Alternatively, the portion with the large diameter may be substantially filled, but also having a channel connected to the first portion.

FIG. 2E shows such a driver variant, mounted to the biomedical device of FIG. 2C. The assembly may further comprise a thread or suture (not shown) for holding the driver and the biomedical device together, e.g. as illustrated in FIG. 14E.

FIG. 3A to FIG. 3C show the assembly of FIG. 2A and FIG. 2B, after the biomedical device is pushed out of the outer tube of the driver. In the example shown, the biomedical device is still in the “delivery configuration”. In practice, or in a variant, the arms and/or the guiding elements may slightly open after the biomedical device is pushed out of the outer tube (see also FIG. 4A to 4C).

FIG. 4A to FIG. 4C show the assembly of FIG. 3A to FIG. 3C after rotation of the guiding elements and the arms. The biomedical device is now in its “deployed configuration”.

FIG. 5A to FIG. 5C show the assembly of FIG. 4A to FIG. 4C after moving the inner tube upwards relative to the outer tube. The biomedical device is still in its “deployed configuration”.

FIG. 6A to FIG. 6C show the assembly of FIG. 5A to FIG. 5C after retracting the arms into the guiding elements. The biomedical device is now in its “retracted configuration”.

FIG. 7A shows a bottom view, and FIG. 7B shows a top view of the assembly of FIG. 6B.

FIG. 8A to FIG. 10F show the biomedical device without the driver.

FIG. 8A to FIG. 8F show the biomedical device of FIG. 1B in its “delivery configuration”.

FIG. 9A to FIG. 9F show the biomedical device of FIG. 8A in its “deployed configuration”.

FIG. 10A to FIG. 10F show the biomedical device of FIG. 9A in its “retracted configuration”.

FIG. 11A to FIG. 11C illustrate an example of how the guiding elements of the biomedical device of FIG. 1B may be configured to rotate relative to the base, and how the arm may be configured to shift or slide relative to the guiding element. FIG. 11A shows the elements individually (e.g. as they may be produced). A thread or suture may be connected or looped through the arm. FIG. 11B shows a relative position of the guiding element and the arm after engagement (e.g. during assembly or sub-assembly). FIG. 11C shows a relative position of the guiding element and the arm after retraction of the arm (e.g. during actual use).

FIG. 12A to FIG. 17B show the assembly and/or the biomedical device relative to a body wall having an illustrative opening.

FIG. 12A to FIG. 12D illustrate the assembly comprising a driver and a biomedical device in its “delivery configuration”, positioned outside of an illustrative opening of a body wall. FIG. 12A is a perspective top view, FIG. 12B is a partially open 3D perspective view, FIG. 12C is a side view, and FIG. 12D is a partially open side view.

FIG. 13A to FIG. 13D illustrate the assembly of FIG. 12A to FIG. 12D, after the outer tube of the driver is partially inserted into the opening of the body wall, and the biomedical device is moved out of the outer tube of the driver. FIG. 13A is a perspective top view, FIG. 13B is a partially open 3D perspective view showing also an inner tube of the driver, FIG. 13C is a side view, and FIG. 13D is a partially open side view.

FIG. 14A to FIG. 14D illustrate the assembly of FIG. 13A to FIG. 13D, after the guiding elements, and thus also the arms are rotated or spread. The biomedical device is in its “deployed configuration”.

FIG. 14E is an illustration showing a possible way how the base (and thus the biomedical device) can be temporarily connected to the inner tube of the driver using a thread or suture.

FIG. 15A and FIG. 15B illustrate the assembly of FIG. 14A to FIG. 14D after the biomedical device is moved towards the body wall, and the penetration means have penetrated in and/or are anchored with inner layers of the wall tissue.

FIG. 16A is a schematic representation of FIG. 15B after pulling the outer tube of the driver out of the opening of the body wall. The biomedical device is still connected to the inner tube of the driver, and the penetration means of the arms are still engaged with the tissue of the body wall.

FIG. 16B shows the biomedical device of FIG. 16A without the body tissue, and furthermore shows a possible way of how the arms can be moved towards each other using threads or sutures.

FIG. 17A shows the biomedical device of FIG. 16B after moving the arms towards each other. The biomedical device is now in its “retracted configuration”.

FIG. 17B shows the schematic representation of FIG. 16A, after moving the arms towards each other, and after disconnecting the driver from the biomedical device.

FIG. 18A shows a bottom view of FIG. 16A.

FIG. 18B shows a bottom view of FIG. 17B.

FIG. 19A to FIG. 21E shows several examples of how a plurality of arms may be mounted to a central component in a manner such that they can rotate (from a substantially axial orientation towards a substantially radial orientation with respect to the central axis), and such that they can subsequently be moved towards each other.

FIG. 19A to FIG. 19C are schematic representations of an embodiment of a biomedical device proposed by the present invention, comprising guiding elements hingedly connected to a base, and arms which can slide with respect to these guiding elements.

FIG. 20A to FIG. 20C are schematic representations of another embodiment of a biomedical device proposed by the present invention, comprising guiding elements connected to the base using living hinges, and comprising arms which can slide with respect to these guiding elements.

FIG. 21A to FIG. 21E are schematic representations of yet another embodiment of a biomedical device proposed by the present invention, comprising arms having an elongated slot or trench or groove, held or clamped between two pillars which are fixedly connected to a base, and which allow the arms to rotate relative to the base, and subsequently allow the arms to move radially with respect to the base. FIG. 21A to FIG. 21C show the arms in side view. FIG. 21D and FIG. 21E show the arms of FIG. 21C from viewing position A-A.

FIG. 22A to FIG. 22G illustrate a second embodiment of a biomedical device proposed by the present invention, having living hinges. FIG. 22A shows a perspective view of the biomedical device in its delivery configuration, connected to a driver. The driver may have an inner tube and outer tube similar as described above, but only the inner tube is shown in FIG. 22A to FIG. 22D. FIG. 22B shows a partially open side view of the biomedical device in its delivery configuration. FIG. 22C shows the second embodiment in an intermediate configuration between the delivery configuration and the deployed configuration. The living hinges of the biomedical device may be configured (e.g. biased) to automatically move the guiding elements to this intermediate orientation after the biomedical device is moved out of the outer tube of the driver. FIG. 22D and FIG. 22E show the second embodiment in its deployed configuration. FIG. 22F shows a partially open side view of the second embodiment in its deployed configuration. FIG. 22G shows a perspective view of the second embodiment in its retracted configuration, but still connected to the driver.

FIG. 23 shows an example of an abdominal wall having several layers.

FIG. 24A shows an example of an arm having penetration means with a barb, as may be used in embodiments of the present invention.

FIG. 24B shows an enlarged perspective view of the penetration means of FIG. 24A.

FIG. 25 illustrates a method in accordance with embodiments.

FIGS. 26A and 26B show a schematic overview of a device in accordance with embodiments, in which a length of the penetration means, e.g. when oriented perpendicular to the elongated arm portion, is limited by substantially half the distance between their respective guiding elements due to a symmetric configuration (e.g. as generally shown in the previous drawings).

FIGS. 27A and 27B show a schematic overview of an alternative configuration of the device, in accordance with embodiments, in which the length of the penetration means can extend over more than half the distance between the guiding elements, by a displacement of opposite penetration means with respect to each other in the axial direction (along the central axis) when in the delivery configuration.

FIGS. 28A and 28B show another alternative configuration of the device, in accordance with embodiments of the present invention, which also allows the length of the penetration means to extend over more than half the distance between the guiding elements, by a displacement of opposite penetration means with respect to each other in a different direction than the axial direction, e.g. in a plane perpendicular to the axial direction. FIGS. 28B and 28C show different embodiments implementing such displacements, in which only the penetration means are offset out of alignment, but not the elongated arm portions supporting the penetration means (FIG. 28B), or in which the entire arms (e.g. elongated arm portions and penetration means) are offset (FIG. 28C).

Furthermore, FIG. 28D shows an arrangement of a plurality (e.g. more than two) of arms using the same principle, in accordance with embodiments of the invention, whereby a claw-like structure is obtained, e.g. by positioning pairs of opposite arms (with respective penetration means) substantially parallel to each other with the penetration means on one side offset to fold in between the opposite penetration means on the other side.

FIG. 29A to 29C show a further embodiment of a device in accordance with embodiments, in a deployed configuration, before and after engagement of the penetration means in a body tissue wall (resp. FIGS. 29A and 29B), and the retracted configuration (FIG. 29C). In this embodiment, the arms are curved, such that the penetration means can move in a continuous motion relative to the base, e.g. by sliding out of the elongated portion of the arm, when engaging into the body tissue.

### DETAILED DESCRIPTION OF ILLUSTRATIVE EMBODIMENTS

The present invention will be described with respect to particular embodiments and with reference to certain drawings but the invention is not limited thereto but only by the claims. The drawings described are only schematic and are non-limiting. In the drawings, the size of some of the elements may be exaggerated and may not be drawn to scale for illustrative purposes. The dimensions and the relative dimensions may not correspond to actual reductions to practice of the invention.

Furthermore, the terms first, second and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

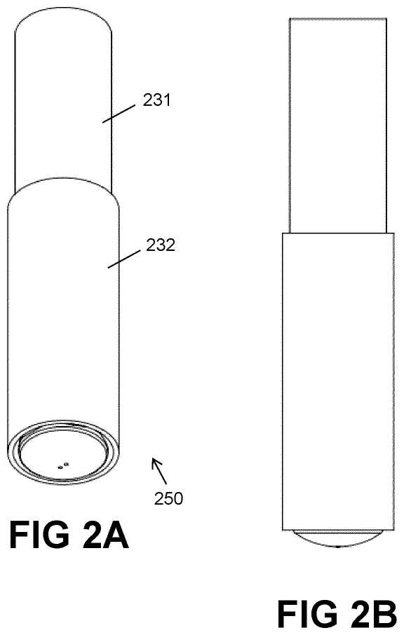

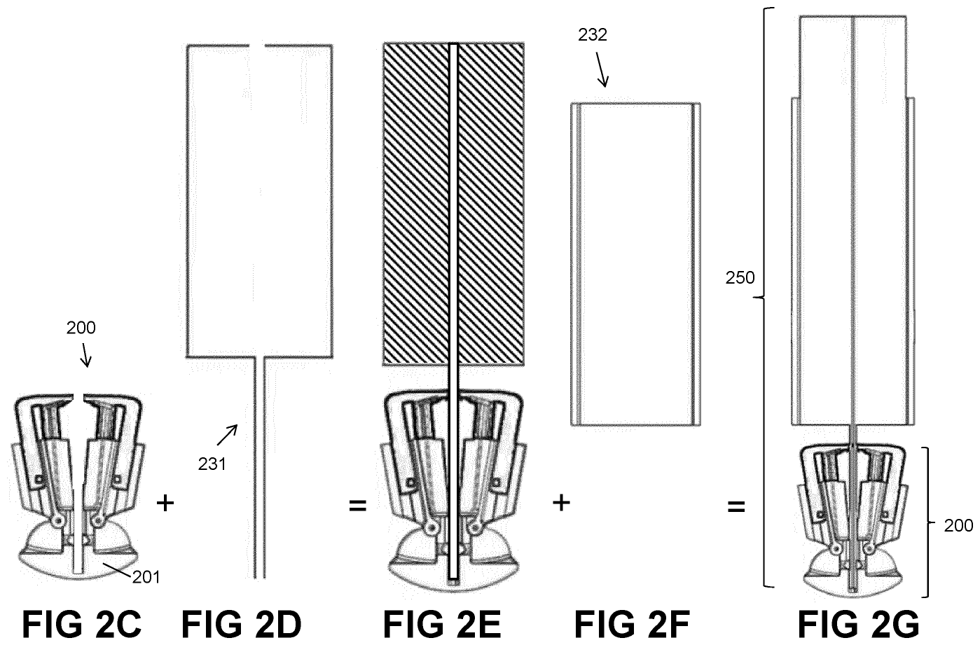

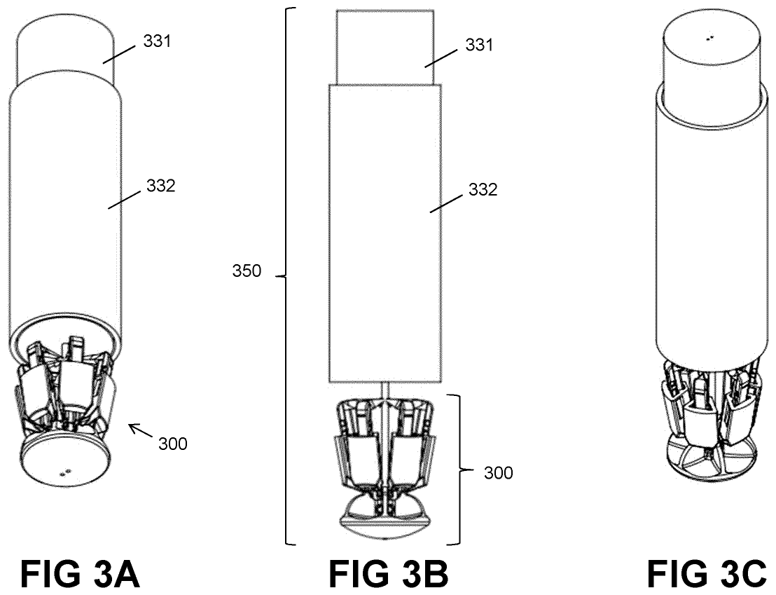

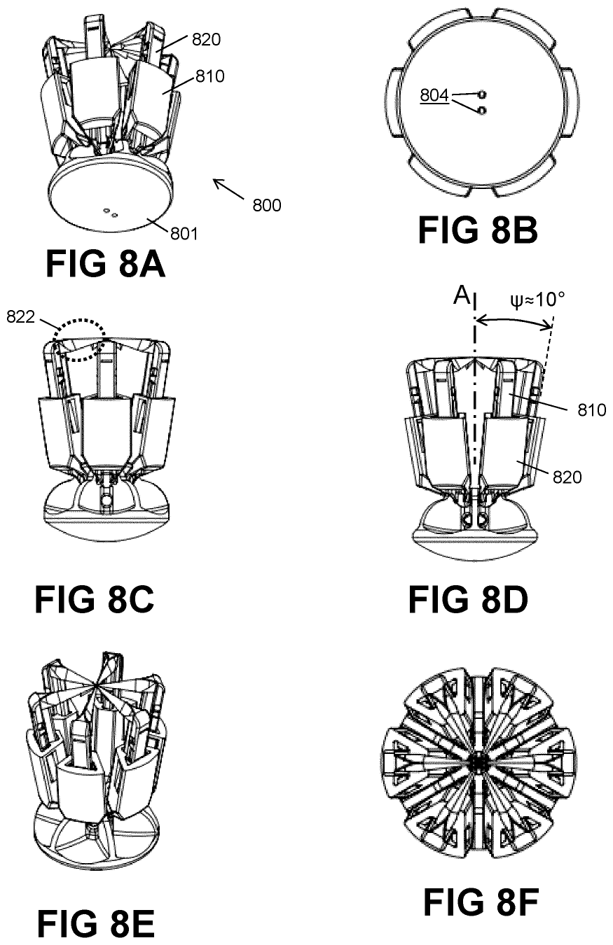

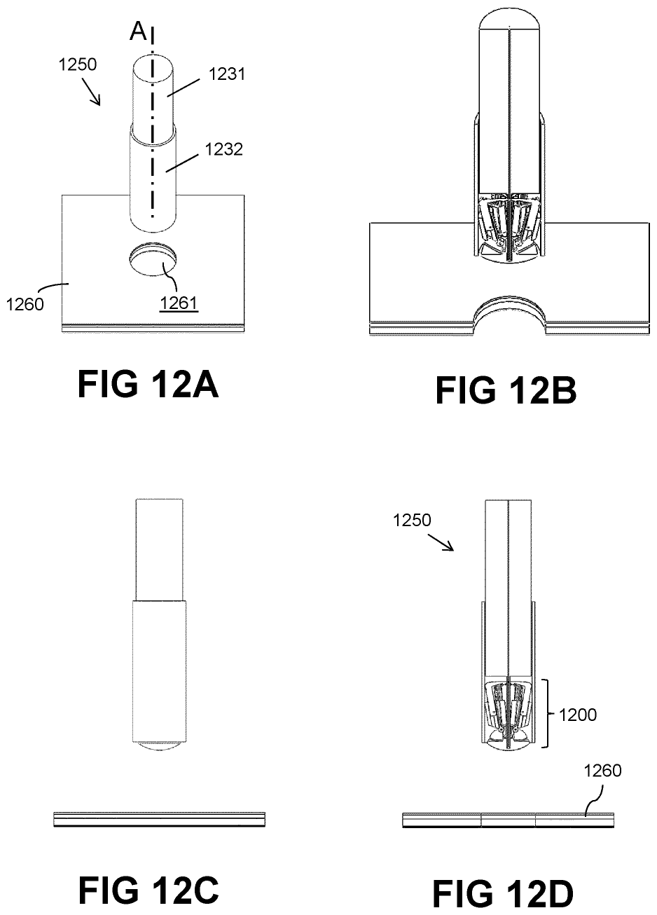

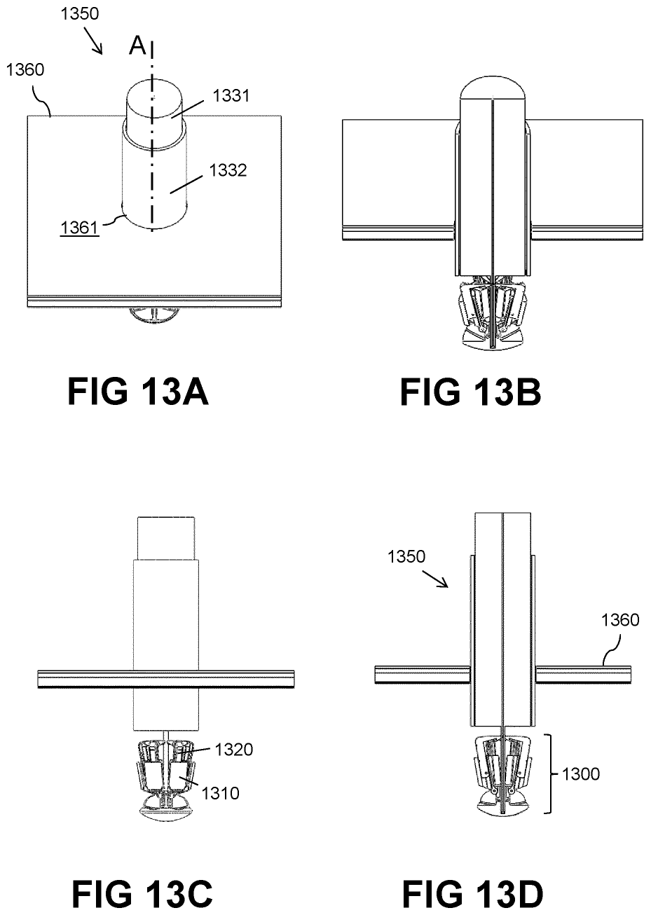

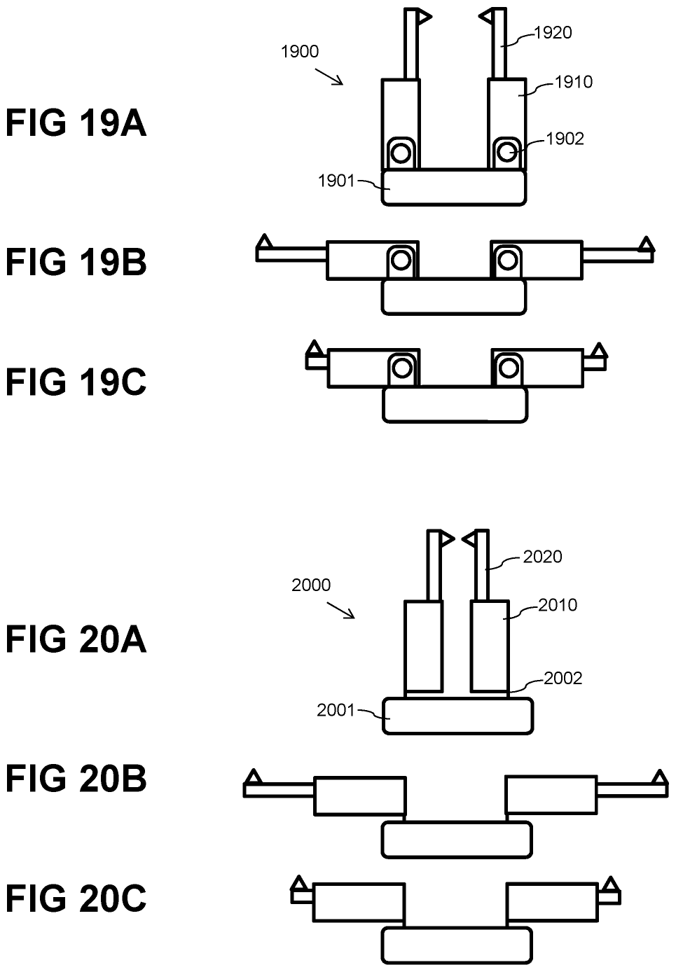

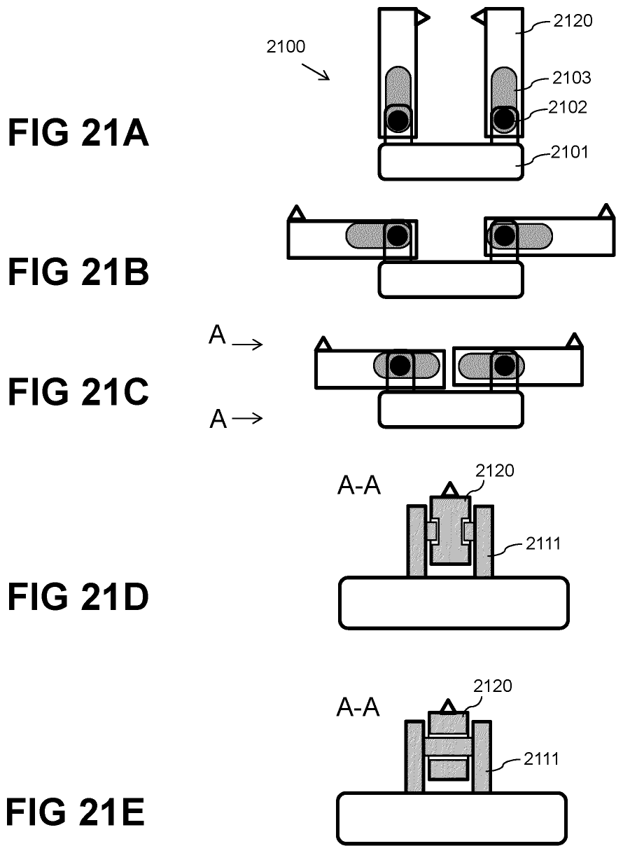

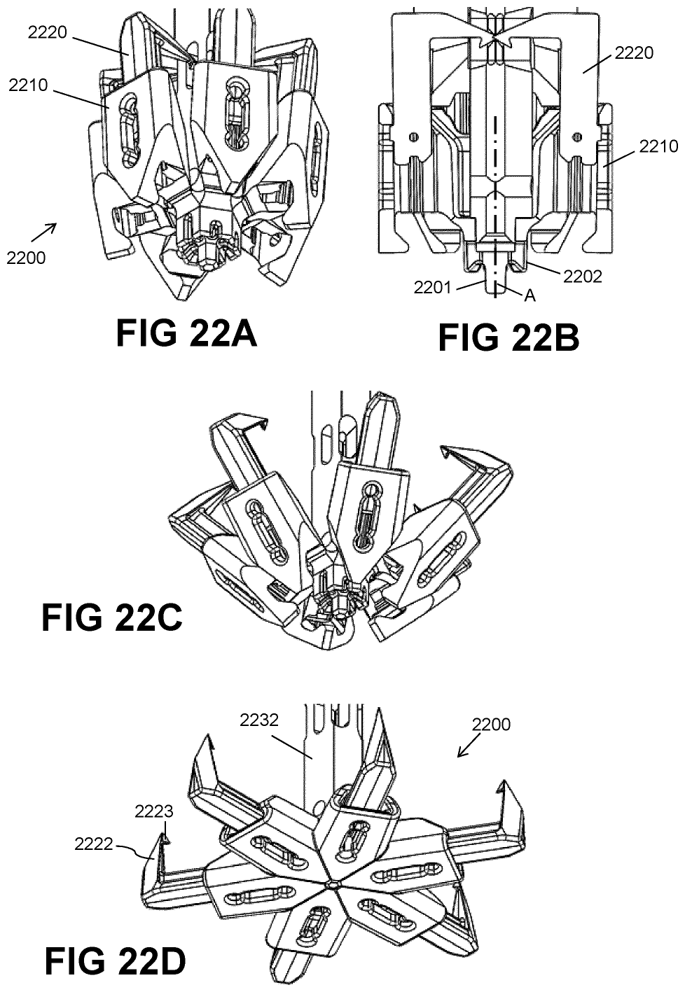

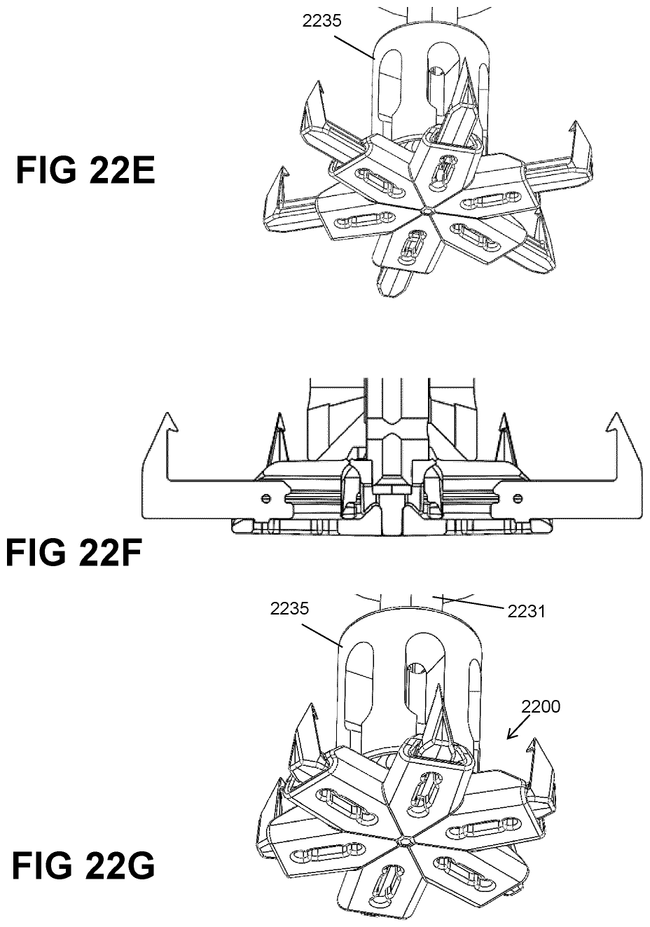

In this document, the expression “delivery configuration” refers to a state of the biomedical device as illustrated for example in FIG. 2A to FIG. 3C (with a driver), or as illustrated in FIG. 8A to FIG. 8F (without a driver), or as illustrated in FIG. 12A to FIG. 13D, (with a driver and relative to a body wall), or as illustrated in FIG. 19A, FIG. 20A, FIG. 21A, FIG. 22A and FIG. 22B.

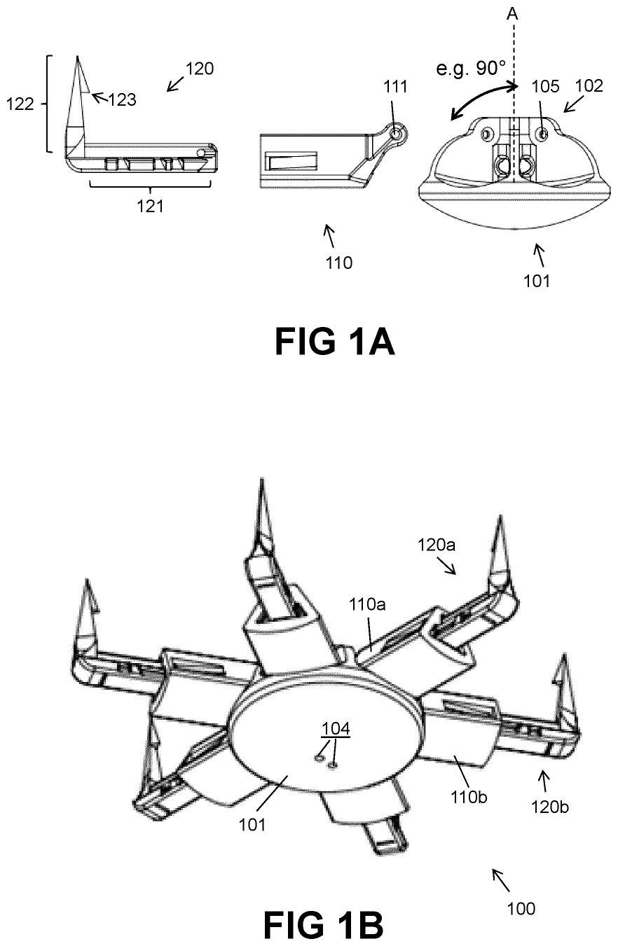

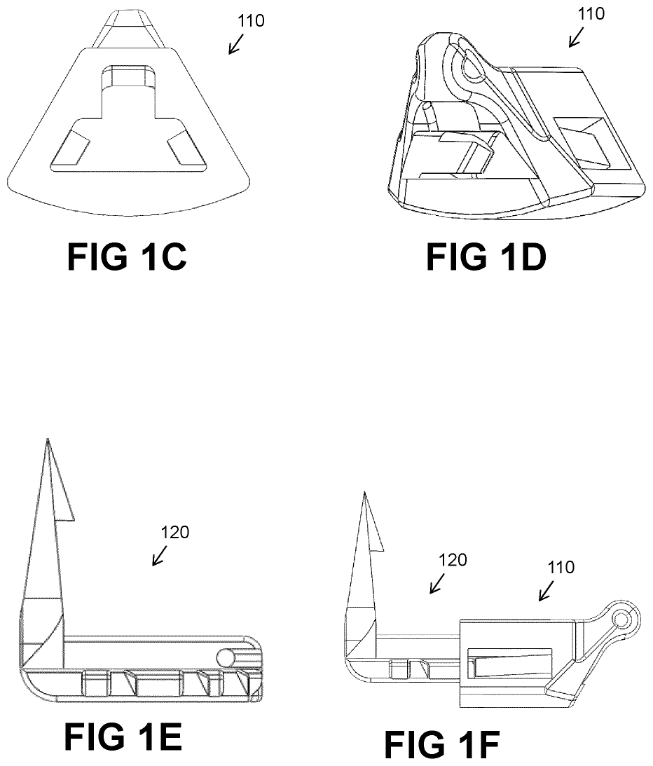

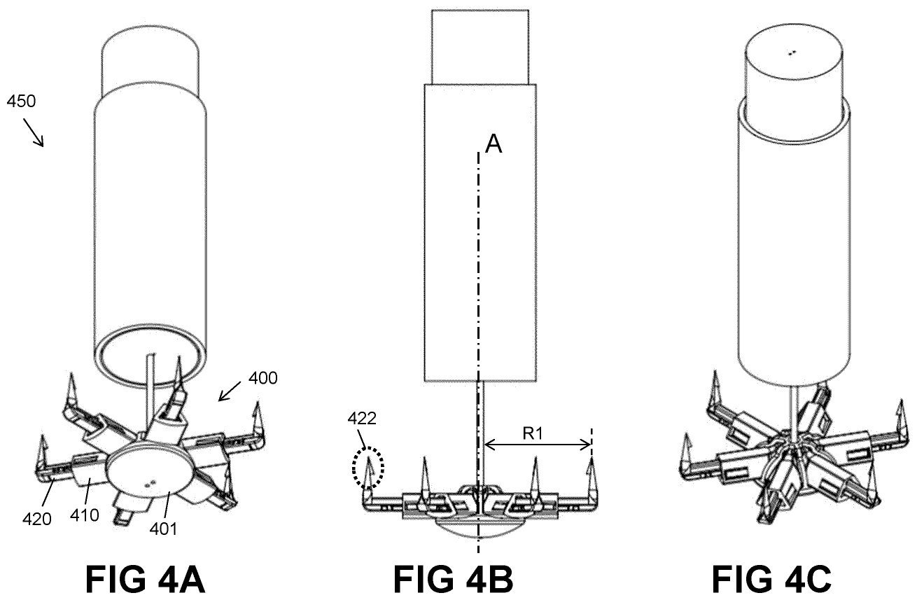

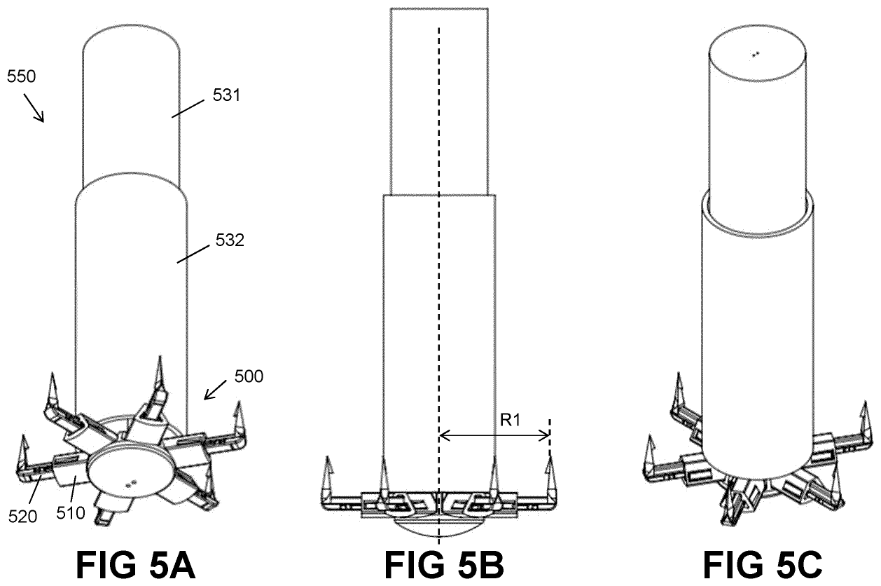

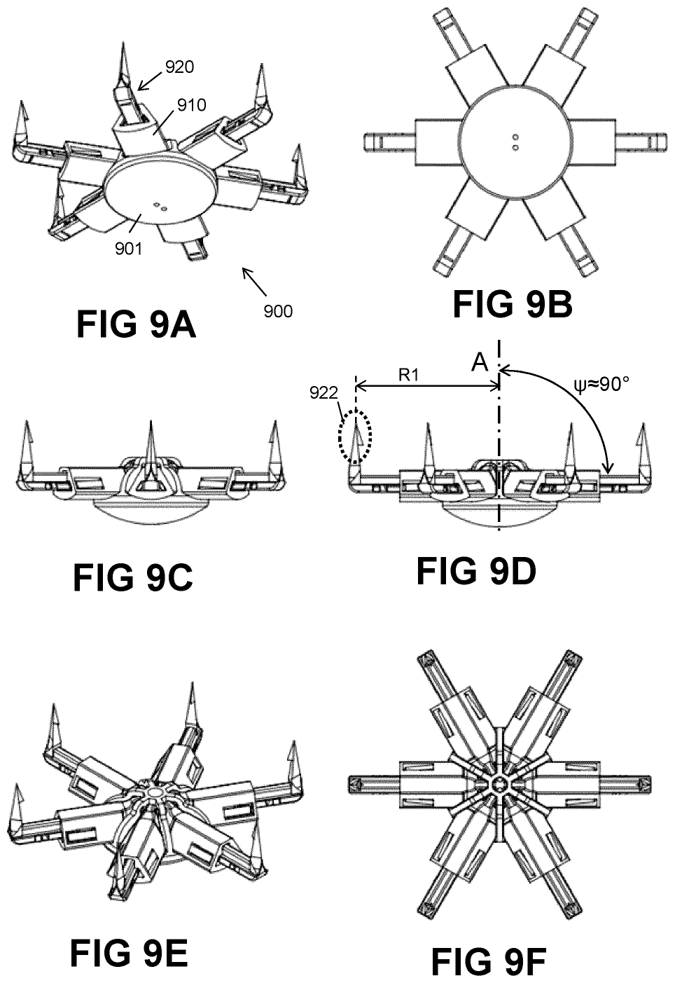

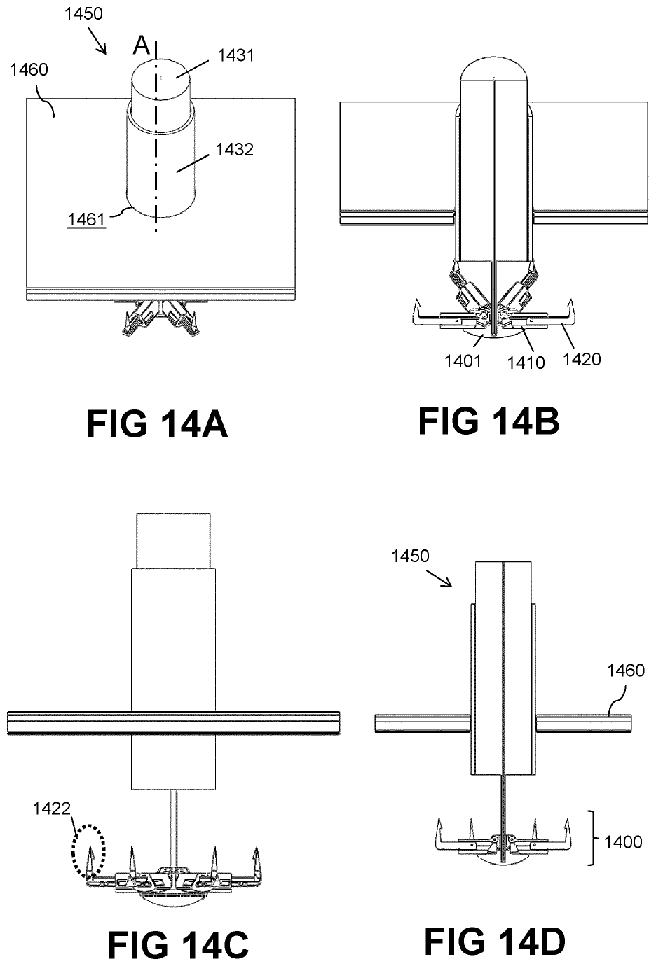

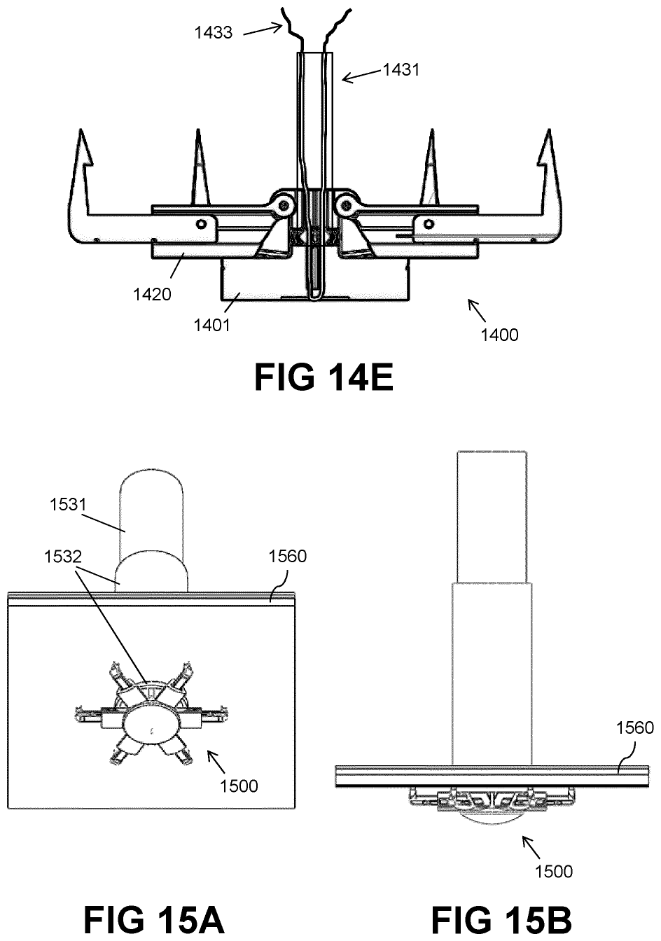

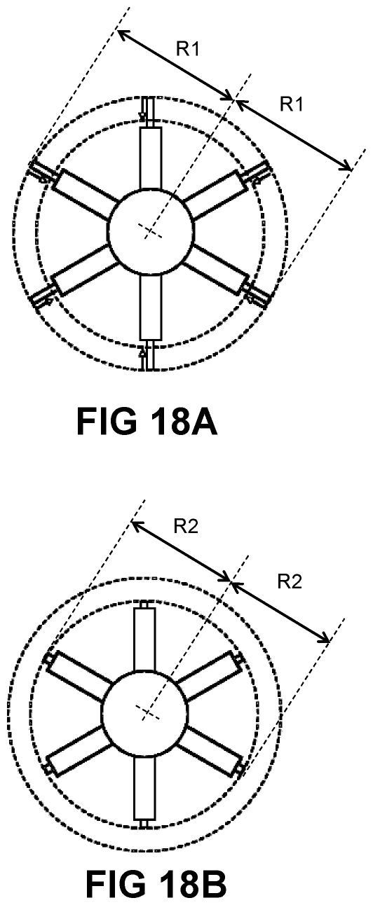

In this document, the expression “deployed configuration” refers to a state of the biomedical device as illustrated for example in FIG. 1B (without a driver), in FIG. 4A to FIG. 5C (with a driver), or as illustrated in FIG. 9A to FIG. 9F (without a driver), or as illustrated in FIG. 14A to FIG. 16B (with a driver and relative to a body wall), or as illustrated in FIG. 18A, FIG. 19B, FIG. 20B, FIG. 21B or FIG. 22D to FIG. 22F.

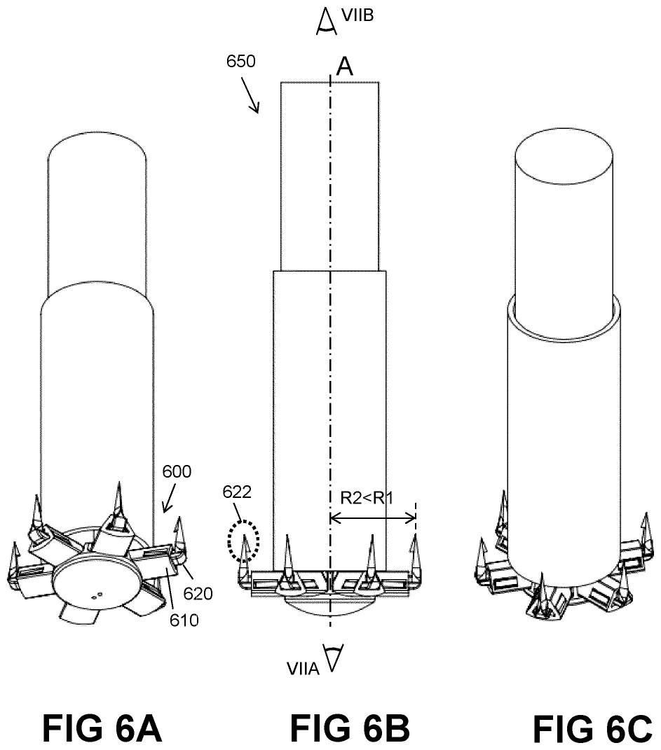

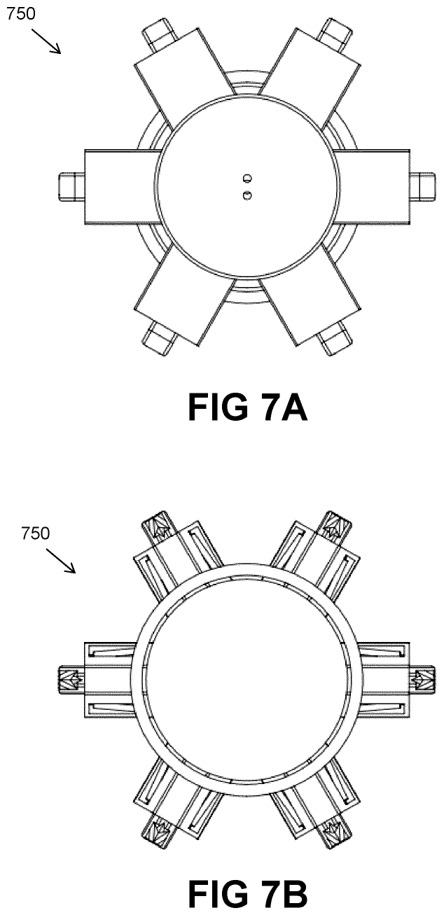

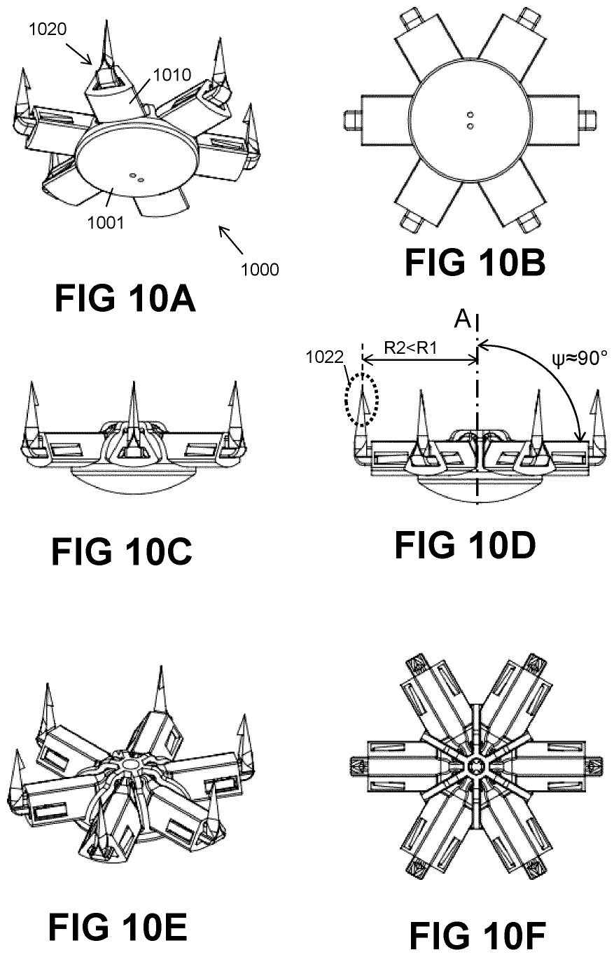

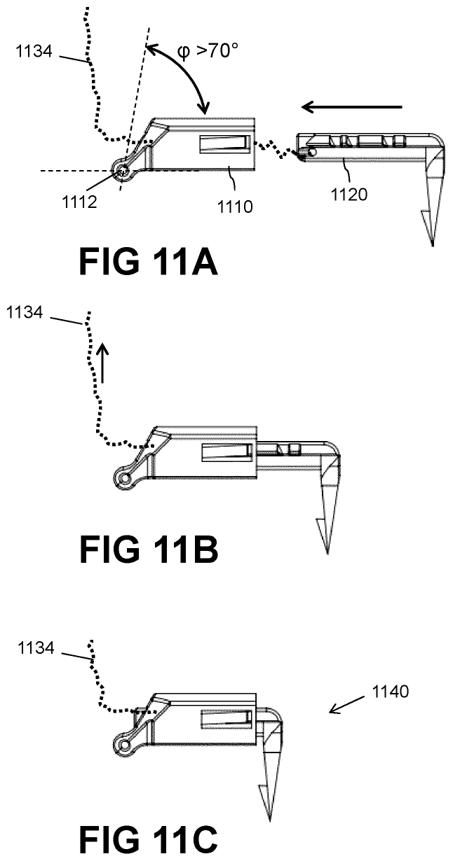

In this document, the expression “retracted configuration” refers to a state of the biomedical device as illustrated for example in FIG. 6A to FIG. 7B (with a driver), or as illustrated in FIG. 10A to FIG. 10F (without a driver), or as illustrated in FIG. 17A, FIG. 17B, FIG. 18B, FIG. 19C, FIG. 20C, FIG. 21C or FIG. 22G.

An object has a “rotation symmetrical shape of order N about an axis” when the object looks exactly the same after rotation over an angle of 360°/N about that axis.

In this document, the terms “central axis” or “reference axis” are used interchangeably.

The present invention relates to implantable biomedical devices and methods for closing or at least reducing the size of an opening of a body wall during or after a minimally invasive surgery (MIS).

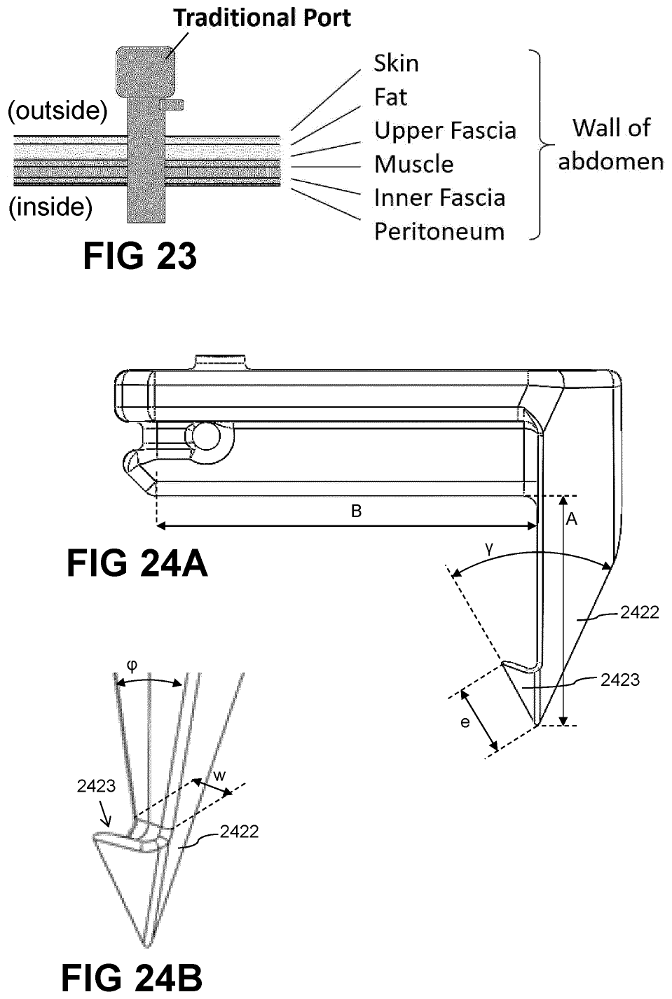

After a MIS procedure is performed, the opening of the body wall needs to be closed, to prevent leakage of body fluids, to prevent infections, or to allow healing. The present invention is partly based on the insight that the body wall typically consists of multiple layers (e.g. as illustrated in FIG. 23), and that it is very difficult to reach and close the inner layers of the wall from the outside of the body, and that hernias may arise if the inner layers are not closed.

The present invention proposes a biomedical device that allows to close or at least allows to reduce the size of an opening of a body wall, in particular at the inner layers thereof.

To this end, the present invention proposes a biomedical device comprising a central component (e.g. a base or a main part), and a plurality of arms (e.g. at least two, or at least three, or at least four, or at least six) movably connected to the central component. The arms may be connected directly to the central component (e.g. by means of clamping elements), or indirectly (e.g. by means of guiding elements). The arms comprise an elongated arm portion (e.g. a portion having a substantially linear shape), and comprise penetration means (e.g. having a tapering end portion, a hook or a sharp tip) for penetrating said body wall. The biomedical device is configured such that a) the elongated arm portions define or form an angle smaller than 20° relative to the central axis when the device is in a delivery configuration; and b) the elongated arm portions are oriented substantially perpendicular to the central axis (A), and a tip of the penetration means is located at a first distance (e.g. R1) from the central axis, when the device is in a deployed configuration; and c) the elongated arm portions are oriented substantially perpendicular to the central axis, and the tip of the penetration means is located at a second distance (e.g. R2) from the central axis, smaller than the first distance, when the device is in a retracted configuration.

It is an advantage that the device has a “delivery configuration” wherein the elongated arm portions form or define an angle smaller than 20° with respect to the central axis, because in this way the biomedical device can have a relatively small outer diameter, which allows the biomedical device to be inserted into the body via a relatively small opening.

It is an advantage that the device has a “deployed configuration” wherein the elongated arm portions are oriented substantially perpendicular to the central axis, or stated in other words, wherein the elongated arm portions are spread, because this allows to position the penetration means at a relatively large distance (e.g. R1) from the central axis.

It is an advantage that the device has a “retracted configuration” wherein the elongated arm portions are still oriented substantially perpendicular to the central axis, but wherein the elongated arm portions are moved towards each other, such that the penetration means are located at a reduced distance (e.g. R2) from the central axis, thereby approaching the body wall and reducing the size of the opening or even closing the opening.

In contrast to earlier solutions aimed at watertight sealing of the opening by means of a membrane, the main purpose of the present invention is to provide a device and a method to approach inner layers of the wall tissue, to thereby close the opening, or at least reduce the size of the opening. This enables faster healing and/or reduces the risk of formation of hernias. After positioning the biomedical device on the inside of the body wall, the top layer, i.e. the skin, can also be approached or closed in known manners, e.g. by stitching.

Referring now to the Figures.

FIG. 1B shows an illustrative example of a biomedical device 100 comprising a central component in the form of a base 101, six guiding elements 110a, 110b, etc. and six arms 120a, 120b, etc. but of course the present invention is not limited thereto, and the number of guiding elements and arms may be less than six, e.g. 2 or 3 or 4 or 5, or may be more than six, e.g. 8 or 10.

The arms 120 comprise an elongated arm portion 121, and penetration means 122.

In preferred embodiments, the biomedical device 100 has a rotational symmetrical shape of an order in the range from 2 to 8 about a central axis A, meaning that the biomedical device looks exactly the same after a rotation over an angle of 360°/N. Preferably the guiding elements 110 and the elongated arm portions 121 are substantially radially oriented when the biomedical device 100 is in the deployed configuration. Preferably the arms 120 are angularly spaced by a constant angle equal to 360° divided by the number of arms, but that is not absolutely required.

The biomedical device of FIG. 1B is shown in its deployed configuration. The various configurations will be described further in more detail.

FIG. 1A shows (from right to left) a base 101, a guiding element 110, and an arm 120 of the biomedical device of FIG. 1B in a exploded view. The base 101 has a first side (at the top of FIG. 1A) with means 102 for connecting the guiding elements 110, and having a second side (at the bottom of FIG. 1A) with a dome shaped surface, which is an example of a smooth surface. This reduces the risk of potential injuries caused by organs contacting the base 101, when inserted in a human or animal body. The base may have two holes (e.g. through openings 104) for connecting a driver, as will be explained further, for example in FIG. 14E.

The guiding elements 110 are hingedly connected to the base 101, for example by means of a dissolvable thread (not shown in FIG. 1A) passing through a mounting opening 105 of the base and through a corresponding mounting opening 111 of the guiding element 110, but other ways of hingedly connecting the guiding elements 110 to the base 101 are also possible, for example by means of a clicking mechanism (not shown) comprising a cavity or a blind hole or a through hole formed in the base, for receiving a protrusion (e.g. a bulge) extending from the guiding element; or comprising a cavity or a blind hole or a through hole formed in the guiding element 110 configured for receiving a protrusion (e.g. a bulge) extending from the base 101.

The guiding elements 110 are preferably rotatable over an angle of at least 70°, e.g. an angle equal to about 70°, or equal to about 80°, or equal to about 90° with respect to the reference axis A (see also FIG. 11A). The base 101 may be made of a solid material, and may be shaped to abut a bottom side of the guiding elements 110 thereby preventing them from rotating over an angle larger than 90°. As can be appreciated from FIG. 1B, the guiding elements 110 can rest on the first side (upper side of FIG. 1A) of the base 101, when the biomedical device 100 is in the deployed or retracted configuration. Thus the base may help to prevent that the guiding elements 110 and/or the arms 120 rotate over an angle larger than about 90° with respect to their initial orientation, namely substantially parallel to the reference axis “A” when the device was in the delivery configuration.

The guiding element 110 may have a tubular portion for receiving an arm portion 121 therein.

The arm 120 may have an overall L-shape comprising a first, elongated arm portion 121, and a second arm portion 122. The first, elongated arm portion 121 is configured for sliding inside the guiding element 110, and is preferably straight. The second arm portion 122 has a tapered shape or an overall pointed shape with a sharp end, so as to form penetration means 122. As can be seen, this overall pointed shape is oriented substantially perpendicular to the elongated arm portion 121, but another angle in the range from about 45° to about 90° may also be used. The penetration means also has a barb 123 for anchoring the penetration means. This will be discussed in more detail in FIG. 24.

In a variant (not shown), the penetration means 122 may not be situated at the end of the elongated arm portion 121, but at a predefined distance from said end. Such an arm may have an overall T-shape.

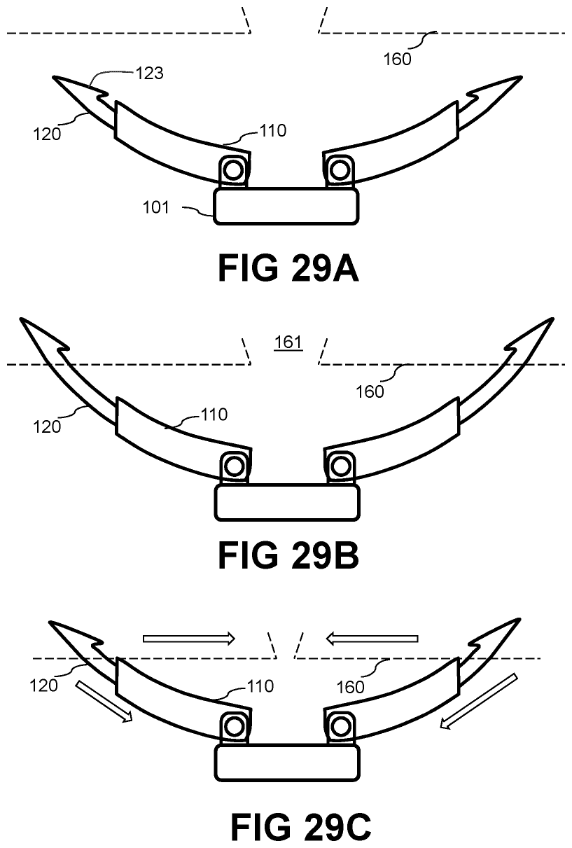

However, embodiments are not necessarily limited to such overall L-shape (or T-shape), which shape is also discussed in more detail further hereinbelow. FIG. 29A to 29C illustrates an alternative embodiment of the device. in which the arms are curved, such that the penetration means can move in a continuous motion relative to the elongated arm portion 121 (and relative to the central component 101. In the deployed configuration (see FIG. 29A), the penetration means can be brought into contact with the body tissue wall 160 to engage the tips of the penetration means into the tissue (see FIG. 29B). The barbs 123 (also discussed more in detail hereinbelow) securely anchor the penetration means into the tissue, e.g. such that the tissue resists a movement of the penetration means opposite to the insertion motion. Then, by retracting the penetration means, the tissue is pulled, by the anchored tips, toward the central axis in order to close (or reduce in size) an opening 161 in the tissue wall, as illustrated by the retracted configuration in FIG. 29C.

Unlike embodiments discussed further hereinbelow (e.g. generally relating to an L-shaped arm shape), in this embodiment, the arms are curved, e.g. both the penetration means 122 and the elongated portion 121 of the arm are curved (along a continuous non-lineal curve, e.g. without corner points along the curve, e.g. a smooth curve, particularly both shaped along the same or a substantially similar curve), such that the penetration means can move in a continuous motion, e.g. slide out of (and into) the elongated portion. This allows the tips to enter the tissue by a piercing motion, before and during engagement of the penetration means 122 in the body tissue wall 160 (resp. FIGS. 29A and 29B), in which this motion has a motion component along the axial direction, while simultaneously having a (radially or laterally) outward directed component. Likewise, the penetration means can move in the opposite direction to achieve the retracted configuration (FIG. 29C), i.e. moving back toward the central axis along the curved path. Since the tips are, in this retraction phase, anchored by the barbs, the retraction motion toward the central axis also pulls (the accompanying axial force component) the central component 101 (e.g. the base) body against the tissue wall, ideally (if sufficient in size) thus covering the remaining opening 161.

It will be appreciated that such “C-shaped” design provides an advantageously simple mechanism in which the (distal ends, e.g. tips, of the) arms are expanded away from the central axis to concomitantly pierce the body wall. The arms merely need to be retracted after engagement to move the central component 101 toward and (ideally) against the tissue wall, while advantageously reducing the size of the opening by the radial pulling action. It is also noted that, whereas the elongated arm portions 121 are oriented substantially perpendicular to the central axis in accordance with embodiments of the invention, when the device is in said deployed configuration, for a curved elongated arm portion this is to be interpreted as referring to the angle of the tangential to the curve in the point where the arm portion joins its support (the central component, hinging point or base) with the central axis, or, alternatively, the mean angle of the elongated arm portion with respect to the central axis (in the deployed configuration).

To continue the more generic description of embodiments (e.g. being not necessarily specific to a particular shape of the arms), FIG. 1C shows a cross section of the tubular portion of the guiding element 110, configured for receiving at least a portion of the elongated arm portion 121. The elongated arm portion 121 may have a cross-sectional shape that fits inside (e.g. is compatible with, or complementary to) a cross-sectional shape of the tubular portion of the guiding element 110. The arm 120 is preferably slidingly connected to the guiding element 110.

Preferably the guiding elements 110 and the arms 120 are provided with a clicking mechanism or a snap-fit mechanism that allow the arms to be engaged with the guiding elements, preferably at least a first time during assembly (when the arm is inserted into the guiding element over a first depth) to prevent that the arm and the guiding element become disengaged; and preferably also a second time (when the arm is inserted into the guiding element over a second depth when the device is brought in the retracted configuration) to prevent that the arms move outwards again, once they are retracted.

FIG. 1D shows a perspective view of a guiding element 110.

FIG. 1E shows a side view of an arm 120. As can be seen, the arm preferably has a shape with structural strength against bending and rotation about its longitudinal axis. In the example, the arm has an overall T-shaped cross section to fit in the substantially T-shaped cavity (see FIG. 1C), but that is not absolutely required, and other cross sections, such as an ellipse, a rectangle, a square or a trapezoidal cross section may also be used.

FIG. 1F shows an example of a subassembly comprising an arm 120 engaged with a guiding element 110. As mentioned above, preferably a clicking mechanism or a snap-fit mechanism prevents the arm 120 and the guiding element 110 from disengaging after assembly.

The biomedical device 100 described above, can be positioned inside a human or animal body using a so-called “driver”. An example of a very simple driver will be described in more detail in FIG. 2D to FIG. 2F, but it is pointed out that the present invention is not limited to any particular driver, and that other suitable drivers can also be used, provided of course that a suitable interconnection or interface is provided to that driver.

As an example, another suitable driver may have a mainly tubular shape, and may comprise two spheres (or balls) which can move radially inwards or outwards when the user pushes a button. In order to connect with such kind of driver, the base of such a biomedical device may comprise a small tube or a central opening having an interior circumferential groove for receiving said spheres.

FIG. 2A shows a perspective view, and FIG. 2B shows a side view of the biomedical device of FIG. 1B, configured in its “delivery configuration”, connected to an illustrative “driver”. The combination of the driver and the biomedical device is typically referred to herein as “assembly” 250. In FIG. 2A and FIG. 2B only the base of the biomedical device is visible, the guiding elements and the arms are located inside the outer tube of the driver.

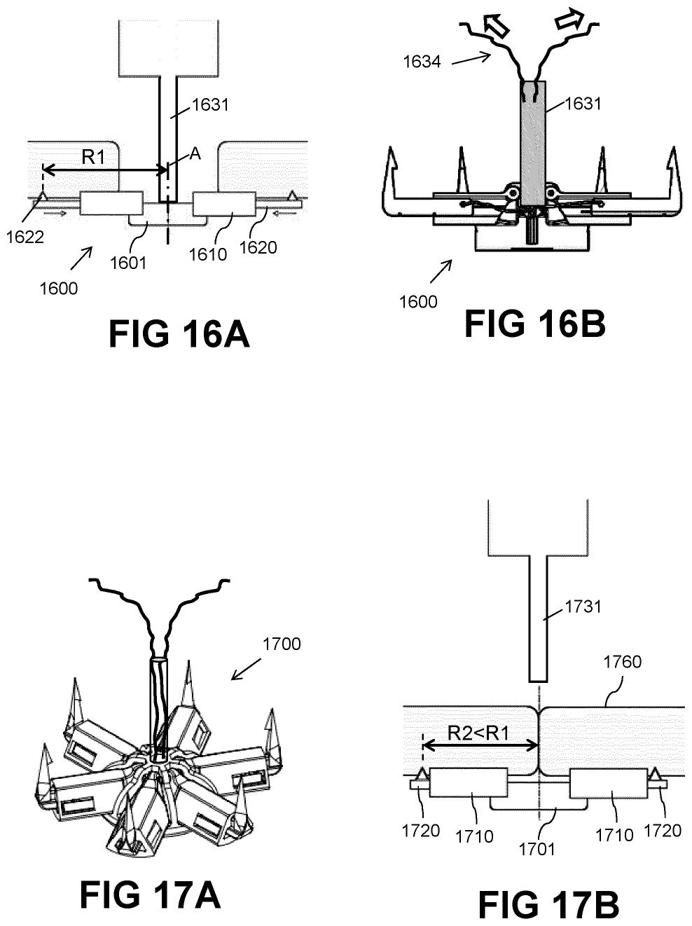

FIG. 2C to FIG. 2G illustrate, by way of example, how the biomedical device 100 of in FIG. 1B can be connected to such a driver to form the assembly 250 of FIG. 2A. The “simple driver” consists of two tubular elements: an inner tube 231 illustrated in FIG. 2D, and an outer tube 232 illustrated in FIG. 2F. As can be seen, the outer tube 232 has a constant diameter, whereas the inner tube 231 has a first portion with a relatively small diameter to engage with the base of the biomedical device, and has a second portion having a diameter slightly smaller than that of the outer tube 232, such that the outer tube 232 can shift over the inner tube 231. The inner tube 231 allows passage of one or more threads or sutures to a space outside the body, as will be further described in FIG. 14E and FIG. 16B.

FIG. 2E shows the biomedical device 200 of FIG. 2C connected to the inner tube 231 of FIG. 2D. In order to establish such a connection, the base 201 may comprise an “interface”, e.g. a cavity or a blind hole for receiving an end of the inner tube 231, more specifically, an end of the first portion thereof having the small diameter (see also FIG. 8F). The base 201 can be connected to the inner tube 231 by means of a thread or suture making a loop through or around the base 201. After the connection, the base, and thus the entire biomedical device 200, can be positioned inside a body through an opening of a body wall by moving the inner tube 231 with the biomedical device 200 connected thereto.

FIG. 2G shows the biomedical device 200 connected to the inner tube 231 of FIG. 2E, with the further addition of the outer tube 232. By comparing FIG. 2A and FIG. 2G, it can be appreciated that FIG. 2A shows the same assembly as FIG. 2G, but the outer tube 232 is slightly shifted relative to the inner tube 231.

It is noted that the inner tube 231 may be a mainly solid component having a channel extending from the top to bottom (as illustrated in FIG. 2E), or may be mainly a hollow component (as illustrated in FIG. 2D). What is important is that the inner tube is sufficiently stiff or rigid for allowing the biomedical device 200 to be positioned inside a body opening, and that it allows passage of one or more threads or sutured, e.g. one thread for connecting/disconnecting the first tube 231 to the base (e.g. as illustrated in FIG. 14E), and one or more threads for pulling the arms inwardly (e.g. as illustrated in FIG. 16B).

FIG. 2C to FIG. 2G show cross-sections of the inner and outer tube 231, 232, and of the biomedical device 200.

FIG. 3B shows a side view of the assembly of FIG. 2G. The assembly 350 comprises a biomedical device 300 and a driver connected thereto. The driver comprises an inner tube 331 and an outer tube 332. The biomedical device 300 is in its delivery configuration.

FIG. 3A and FIG. 3C show a perspective view of the assembly 350. By comparing FIG. 2A and FIG. 3A, it can be appreciated that the assembly 350 of FIG. 3A can be obtained by slightly shifting the outer tube 232 of the assembly 250 of FIG. 2A relative to the inner tube 231.

FIG. 4A to FIG. 4C show the assembly of FIG. 3A to FIG. 3C after rotating the guiding elements 410, and along with the guiding elements, also the arms 420 relative to the central axis. This may be achieved for example by shaking or rotating the biomedical device 400 about its central axis A (due to centrifugal force), or by using a spring, or by using a biasing force related to the elasticity of the material or in any other suitable manner causing the guiding elements 410 to rotate relative to a reference axis A from an original orientation defining or forming an angle L of about 10° relative to the reference axis A (as in FIG. 8D), to an orientation defining or forming an angle LP of about 90° with the reference axis (as in FIG. 9D). In the example shown in FIG. 4A to FIG. 4C, the guiding elements 410 cannot rotate further than 90°, because they will abut the upper surface of the base 401. The biomedical device 400 is now in its “deployed configuration”. The rotation of the guiding elements 410 will also cause the penetration means 422 to be rotated. The tip of the penetration means 422 is situated at a first radial distance R1 from the central axis A. In the example shown, the biomedical device 400 has six arms, the arms are uniformly angularly spaced, the tips of the penetration means 422 are located on a virtual circle with radius R1 (see also FIG. 18A).

FIG. 5A to FIG. 5C show the assembly of FIG. 4A to FIG. 4C after moving the inner tube 531 of the driver upwards relative to the outer tube 532 of the driver. The biomedical device 500 itself remains in its “deployed configuration”. The reason for this move will become clear further (e.g. in FIG. 15A).

FIG. 6A to FIG. 6C show the assembly of FIG. 5A to FIG. 5C after retracting the arms 620 into the guiding elements 610, for example radially inwards with respect to the central axis A. Preferably all the arms 620 are retracted, but that is not absolute required for the invention to work, and it suffices that only some of the arms 620 are retracted. The retraction can be achieved for example by pulling one or more threads or sutures 1134, 1634 connected to the arms 620 and guided through the guiding elements 610, e.g. as illustrated in more detail in FIG. 11B, FIG. 11C and FIG. 16B. The tips of the penetration means 622 of FIG. 6A to FIG. 6C are now situated at a second radial distance R2 from the central axis A, smaller than the first radial distance R1. The tips are located on a virtual circle with radius R2 (see also FIG. 18B). The biomedical device 600 is now in its “retracted configuration”.

FIG. 7A shows a bottom view, and FIG. 7B shows a top view of the assembly 650 of FIG. 6B.

FIG. 8A to FIG. 10F show various views on the biomedical device without the driver.

FIG. 8A to FIG. 8F show the biomedical device 800 in the “delivery configuration”. In this configuration, the arms 820 (or more specifically the elongated arm portions thereof) define or form an angle ψ smaller than 20° relative to the reference axis A, or smaller than 15°, e.g. equal to about 10°; and the penetration means 822 are oriented towards the reference axis A. As can best be seen in FIG. 8D and FIG. 8F, there is a room for receiving a small cylindrical object (e.g. the first tube 231 of a driver) between the various guiding elements 810 and between the various arms 820. As can best be seen in FIG. 8B and FIG. 8F, the base 801 may have two holes (or through openings) for allowing passage of a thread or suture 1433 for releasably connecting the first tube 231 to the base 801, (see also FIG. 14E).

FIG. 9A to FIG. 9F show the biomedical device of FIG. 8A to FIG. 8F in the “deployed configuration”. The arms 920, or rather the elongated arm portions thereof, define or form an angle F of about 90° relative to the reference axis A; and the penetration means 922 are oriented substantially parallel to the reference axis A, pointing away from the base 901. The tips of the penetration means 922 are situated at a first radial distance R1 from the reference axis A.

FIG. 10A to FIG. 10F show the biomedical device of FIG. 9A to FIG. 9F in the “retracted configuration”. The arms 1020, or rather the elongated arm portions thereof, still define or form an angle ψ of about 90° relative to the reference axis A; and the penetration means 1022 are still oriented substantially parallel to the reference axis A, pointing away from the base 1001, but the tips of the penetration means 1022 are situated at a second radial distance R2, smaller than the first radial distance R1, from the reference axis A.

FIG. 11A to FIG. 11C illustrate that the guiding elements 1110 may be pivotally mounted to a base (e.g. by connecting an opening 105 of the base (see FIG. 1A) with an opening 1112 of the guiding element 1110 using a suture or a thread (not shown), and that the arm 1120 may be slidingly mounted to the guiding elements 1110, e.g. by inserting the arm into the guiding element. In preferred embodiments, the base and the guiding elements 1110 may be configured to rotate over an angle ϕ of about 70° to 90° when moving from the “delivery configuration” to the “deployed configuration”. The arm 1120 may be moved relative to the guiding elements 1110 by pulling one or more threads or sutures 1134. In an embodiment, each arm is connected to an individual thread or suture. In other embodiments, at least two arms are connected to a common thread, which is looped through an opening of said at least two arms.

FIG. 11A shows a single guiding element 1110 and a single arm 1120 before they are assembled. FIG. 11B shows a relative position of the guiding element 1110 and the arm 1120 after engagement. The guiding element 1110 and the arm 1120 may have clicking means or snap fitting or the like to prevent separation of the arm after assembly. FIG. 11C shows a relative position of the guiding element 1110 and the arm 1120 after retraction of the arm. The guiding element 1110 and the arm 1120 may have further clicking means or snap fitting or the like to prevent the arm from moving outwardly once it has been retracted.

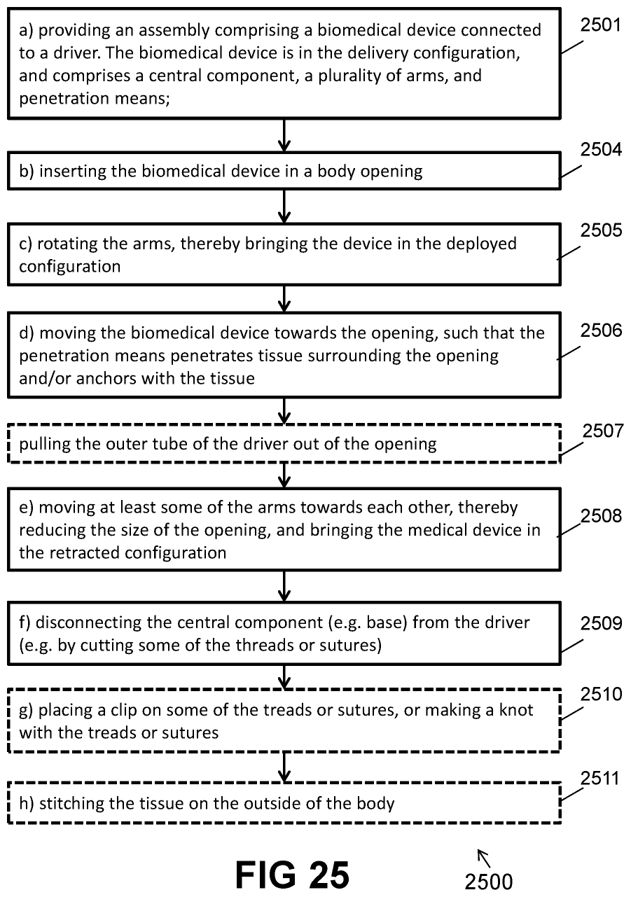

FIG. 12A to FIG. 17B show the biomedical device described above, as it may be used relative to a body wall 1260. These drawings also illustrate different steps of a method of closing an opening 1261 of a wall of a human or animal body using a biomedical device (or an assembly comprising such a biomedical device) as described above. FIG. 25 shows a flowchart of such a method.

FIG. 12A to FIG. 12D illustrate an assembly 1250 comprising a driver and a biomedical device 1200 in its “delivery configuration” (similar as in FIG. 2A and FIG. 2B). The assembly 1250 is positioned outside of an illustrative opening 1261 of a body wall 1260. FIG. 12A is a perspective top view, FIG. 12B is a partially open 3D perspective view, FIG. 12C is a side view, and FIG. 12D is a cross-section view.

FIG. 12A to FIG. 12D illustrate a typical situation after performing step 2501 (see FIG. 25) of “providing an assembly comprising a biomedical device connected to a driver”.

In a next step 2504, the biomedical device 1200 (and a portion of the driver) will be inserted into the opening 1261 of the body wall 1260.

FIG. 13A to FIG. 13D are illustrations of the assembly of FIG. 12A to FIG. 12D after the outer tube 1332 of the driver is partially inserted in the opening 1361 of the body wall 1360, and the biomedical device 1300 is moved out of the outer tube 1332 of the driver (similar as in FIG. 3A to FIG. 3D, and FIG. 8A to FIG. 8F), into a cavity of the body.

FIG. 13A is a perspective top view, FIG. 13B is a partially open 3D perspective view showing also the inner tube 1331 of the driver, FIG. 13C is a side view, and FIG. 13D is a partially open side view.

FIG. 13A to FIG. 13D illustrate the situation after performing step 2504 (see FIG. 25) of “inserting the biomedical device in a body opening”.

In a next step 2505, the arms will be rotated outwardly (or “spread”).

FIG. 14A to FIG. 14D are illustrations of the assembly of FIG. 13A to FIG. 13D after the guiding elements, and thus also the arms are rotated away from the central axis (or spread). This can be achieved for example by shaking the assembly, or by rotating the assembly around its longitudinal axis (due to centrifugal force), or by using spring elements (not shown), or in other suitable ways. The guiding elements 1410 may abut an upper side of the base 1401 of the biomedical device 1400, such that elongated portions of the arms 1420 are oriented substantially perpendicular to the reference axis A, and the penetration means 1422 are oriented towards the body wall. A tip of the penetration means 1422 is located at a first radial distance R1 from the reference axis A. The biomedical device 1400 is now in its “deployed configuration”, similar as in FIG. 4A to FIG. 4D, and FIG. 9A to FIG. 9F.

FIG. 14A to FIG. 14D illustrate the situation after performing step 2505 of FIG. 25 of “rotating the arms, thereby bringing the device in the deployed configuration”.

In a next step 2506, the biomedical device 1400 will be moved back towards the opening 1461 in the body wall 1460, (i.e. upwards in FIG. 14A to FIG. 14D), thereby causing the penetration means 1422 to penetrate tissue surrounding the opening 1461. If the penetration means also comprises a barb (as will be explained further in FIG. 24A and FIG. 24B), this movement also causes anchoring of the biomedical device.

FIG. 14E is an illustration showing in more detail a possible way how the base 1401 of the biomedical device 1400 can be temporarily or releasably connected to the inner tube 1431 of the driver (only a portion of which is illustrated in FIG. 14E) using a thread or a suture 1433 that forms a loop through or around the base 1401. This picture also shows in more detail that the biomedical device 1400 has some space available for receiving or accommodating an end of the inner tube 1431 of the driver. This “space” may comprise an empty space or a blind hole or the like between various guiding elements 1410 and/or in the base 1401 itself. Such provisions for allowing a temporal connection of the base to the driver, e.g. a blind hole, is also referred to herein as “an interface to an external device”.

FIG. 15A and FIG. 15B are illustrations of the assembly of FIG. 14A to FIG. 14D after the biomedical device is moved (e.g. pulled back) towards the body wall 1560, and the penetration means (e.g. pins) have penetrated and/or are anchored with the wall tissue. This may be achieved by pulling the driver upwards. The biomedical device 1500 itself is still configured in the “deployed configuration”, and the tips of the penetration means are still at a first distance R1 from its reference axis A.

FIG. 15A and FIG. 15B illustrate the situation after performing step 2506 of FIG. 25 of “moving the biomedical device towards the opening”. In a next step 2507, depending on the kind of driver that is being used, the outer tube 1532 of the driver may be pulled out of the opening.

FIG. 16A is a schematic representation of FIG. 15B after pulling the outer tube 1532 of the driver out of the opening 1661 of the body wall 1660. The biomedical device 1600 itself is still connected to the inner tube 1631 of the driver, e.g. by means of a thread or suture (e.g. as shown in FIG. 14E), and the penetration means 1622 (e.g. pins) of the arms 1620 are still engaged with the tissue of the body wall, and are located at a radial distance R1 from the central axis A.

FIG. 16A shows the situation after performing step 2507 of FIG. 25 of “pulling the outer tube of the driver out of the opening”. In a next step 2508 (see FIG. 25), the arms 1620 of the biomedical device 1600 will be moved (e.g. pulled) towards each other for closing the opening, or at least reducing the size of the opening.

FIG. 16B shows the biomedical device 1600 of FIG. 16A without the body tissue, and furthermore shows a possible way of how the arms 1620 can be moved towards each other by pulling one or more threads or sutures 1634 which are connected to, or looped through one or more arms, and which are guided through the driver. Preferably dissolvable or bioabsorbable sutures are used.

FIG. 17A shows the biomedical device 1600 of FIG. 16B after performing step 2508 of “moving at least some of the arms towards each other”, but before disconnection the biomedical device from the driver. The biomedical device 1700 itself is now in its “retracted configuration”.

FIG. 17B shows the schematic representation of FIG. 16A, after moving the arms 1620 towards each other, and after disconnecting the inner tube 1631 of the driver from the base 1601, and removing the driver out of the opening of the body wall. A radial distance between a tip of the penetration means and the reference axis A of the biomedical device is reduced to a second distance R2 smaller than the first distance R1 (see also FIG. 10D). This will cause the opening to be closed from the inside of the body, or at least to approach inner layers of the tissue of the body wall in order to reduce the size of the opening. By doing so, the wound can typically heal faster, and the risk of the formation of hernias can be reduced. The position of the arms 1720 relative to the guiding elements 1710 may be locked by means of a clicking mechanism or a snap-fit mechanism or the like, in order to prevent that the arms move outwardly after being retracted.

FIG. 18A shows a bottom view of FIG. 16B showing the biomedical device 1600 in the “deployed configuration”. In the example shown, tips of the penetration means are located on a virtual circle having a radius R1.

FIG. 18B shows a bottom view of FIG. 17A, showing the biomedical device 1700 in the “retracted configuration”. Tips of the penetration means are located on a virtual circle having a radius R2 smaller than R1. It can be understood from FIG. 18A and FIG. 18B that the tissue around the opening of the body wall is approached, in particular the inner layers of the body wall, and that the size of the opening is reduced.

FIG. 19A to FIG. 21E show various mechanisms which can be used by biomedical devices of the present invention, comprising a plurality of arms that can be rotated and retracted. Some embodiments comprise guiding elements, other embodiments do not use guiding elements. Some embodiments use rigid hinges, other embodiments use living hinges, some embodiments don't use hinges at all. FIG. 19A to FIG. 21E are mainly focused on the mechanisms for moving the arms.

FIG. 19A to FIG. 19C are schematic representations of a biomedical device 1900 comprising guiding elements 1910 hingedly connected to a central component 1901 (e.g. a base), and having arms 1920 which can slide with respect to (e.g. inside of) the guiding elements 1910.

FIG. 20A to FIG. 20C are schematic representations of a biomedical device 2000 comprising guiding elements 2010 connected to a central component 2001 (e.g. base) by means of living hinges 2002, and comprising arms 2020 which can slide with respect to (e.g. inside of) the guiding elements 2010.

FIG. 21A to FIG. 21E are schematic representations of a biomedical device 2100 not comprising guiding elements, but comprising a plurality of arms 2120 having an elongated slot or trench or groove 2103 (indicated in dark gray). The arms are held or clamped between two pillars 2111 fixedly connected to the central component 2101 (e.g. base). The elongated slots or trenches or grooves 2103 are configured for allowing the arms 2120 to rotate relative to the base 2101 when oriented in a range from a first orientation substantially parallel to the reference axis A (FIG. 21A) to a second orientation substantially perpendicular to the reference axis (FIG. 21B). When in the second orientation, the arms may be moved in a radial direction. FIG. 21A to FIG. 21C show the elongated portion of the arms in side view. FIG. 21D shows a base with two pillars 2111, each pillar having a protrusion, and shows an arm 2120 having a groove clamped between the protrusions. FIG. 21E shows a base with two pillars 2111, and a cylindrical element interconnecting the pillars, and shows an arm 2120 having an elongated opening, the cylindrical element passes through said elongated opening, thereby holding the arms.

FIG. 22A to FIG. 22G illustrate a second embodiment of a biomedical device 2200 proposed by the present invention, having living hinges. This second embodiment can be seen as a variant of the first embodiment described above, with the following main differences: (1) the second embodiment does not have a base with a dome-shape but has a central component 2201 with a central axis A; (2) the guiding elements 2210 are connected to the central component by means of living hinges, and are integrally formed with the central component; (3) the guiding elements can also rotate from a first orientation substantially parallel to the central axis, but in this embodiment, they can rotate until they abut other guiding elements 2210 rather than a top surface of the base; (4) this biomedical device 2200 also has a smooth surface when it is in the deployed or retracted configuration, not because of a base component having a dome-shaped surface, but because a relatively smooth surface is formed by the guiding elements 2210 when arranged in an edge-to-edge arrangement, e.g. as illustrated in FIG. 22D.

Everything else described above is also applicable here, mutatis mutandis, for example: the arms 2220 can slide relative to (e.g. inside) the guiding elements 2210, the arms 2220 can be moved towards each other by means of threads or sutures; the arms may have an overall L-shape or T-shape; the arms have penetration means 2222 for penetrating the body wall, tips of the penetration means are situated at a first radial distance R1 when in the deployed configuration, and are situated at a second radial distance R2 smaller than the first radial distance R1, when in the retracted configuration; the penetration means 2222 preferably further comprise a barb 2223 for anchoring the biomedical device; this biomedical device 2200 can also be connected to a driver, etc.

This second embodiment can also be used to close an opening of a body wall, using the method of, or a method very similar to that shown in FIG. 25.

The main advantages of the second embodiment are: (i) the central component and the guiding elements are integrally formed, which means that the assembly of this device requires less handling; (ii) the living hinges may be designed such that the guiding elements are biased outwardly when the device is in the delivery configuration, which means that the device does not have to be shaken or rotated about its central axis for opening or spreading the guiding elements.

It is noted that the driver for the second embodiment may comprise an additional tubular element 2235 (see FIG. 22G), which can be shifted over the first tubular element 2231 of the driver, and which can be used to contact an upper side of the guiding elements 2210 for opening them.

Since the second embodiment of the biomedical device functions very much in the same way as the first embodiment, it suffices to say that FIG. 22A shows a perspective view of the device in its delivery configuration, connected to a driver, FIG. 22B shows a partially open side view of the device in its delivery configuration, FIG. 22C shows the second embodiment in an intermediate configuration between the delivery configuration and the deployed configuration, FIG. 22D and FIG. 22E show the second embodiment in its deployed configuration, FIG. 22F shows a partially open side view of the second embodiment in its deployed configuration, and FIG. 22G shows a perspective view of the second embodiment in its retracted configuration, and still connected to the driver.

FIG. 23 shows an example of an abdomen wall having multiple layers, and shows a traditional port inserted in an opening of the body wall. The main purpose of this figure is to show that it is beneficial but challenging to close the inner layers of the body wall solely from the outside. The present invention provides a solution to that problem.

FIG. 24A shows an example of the shape and size of a particular arm having penetration means 2422 with a barb 2423. Many tests were performed for optimizing the design of this particular arm for closing an opening caused by a 10.0 mm trocar used during laparoscopic surgery. The following set of dimensions were found to be particularly beneficial for anchoring the biomedical device with an abdominal wall of an human being: A=3.2 mm±25%, e=0.5 mm to 2.0 mm, γ=55°±15°, and w=0.35 mm±50%. But of course, the present invention is not limited to these particular dimensions, and other dimensions may be used as well, especially when the biomedical device is used for other applications.

FIG. 24B shows an enlarged perspective view of the penetration means of FIG. 24A. It has an overall tapering shape with a sharp tip for allowing penetration of a body wall. In preferred embodiments of the present invention, the penetration means 2422 furthermore comprises a barb 2423, which offers the advantage that the biomedical device can be anchored in the wall, and reducing the risk of involuntarily disengagement.

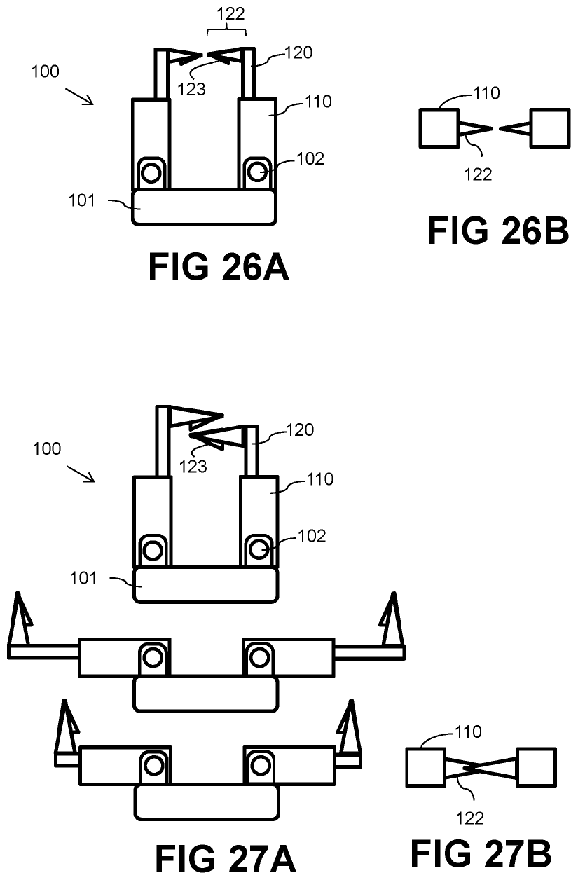

As discussed hereinabove, the arms may have an overall “L”-shape (or equivalent shape with a corner, e.g. a “T” shape). For example, the penetration means may be (e.g. substantially) perpendicular to the elongated portion of the arm. However, it will be seen that in a symmetric arrangement, e.g. as illustrated throughout drawings discussed hereinabove, the length of the penetration means may be limited by substantially half of the distance between hinging points of the arms (e.g. of the distance between their corresponding guiding elements). This is illustrated in FIGS. 26A (side view) and 26B (top view). As can be seen, the length of the penetration means 122 (e.g. when oriented substantially perpendicular to the elongated arm portion) is limited by (substantially) half the distance between their respective connection means 102 (e.g. hinging points) due to the symmetric arrangement of the arms. The diameter of the device in the folded, “delivery” configuration (which preferably is kept minimal) thus appears to limit the available length of the penetration means. Nonetheless, a longer length (for a same, preferably minimal, diameter) of the penetration means may be preferable, e.g. to be able to penetrate deeper into the tissue, and thus to have a firmer grip on the tissue. It is noted that the alternative “corner-less” embodiment, e.g. as shown in FIG. 29 and already discussed hereinabove, may also provide an advantageously longer length of the penetration means relative to the diameter of the device (particularly and advantageously, in that case, the available length is not substantially affected by the diameter at all).

However, this limitation can also be overcome or alleviated for L-shaped and similar constructions. For example, FIGS. 27A and 27B show an alternative configuration of the device, in which the length of the penetration means can extend over more than half the distance between the hinging points, by a displacement of opposite penetration means 122 (e.g. of arms arranged opposite to each other with respect to the central axis), when the device is in the delivery configuration. While one arm of the pair of opposite arms may be shorter than the other to accommodate this asymmetry, this is not strictly necessary. For example, the guiding elements may be adapted to position the penetration means (of the opposite arms) at different axial positions when the device in the delivery configuration, such that they overlap (cf. the top view in FIG. 27B), while allowing the penetration means to move to symmetrical positions in the deployed and/or retracted confiugrations (cf. middle and bottom illustration in FIG. 27A).

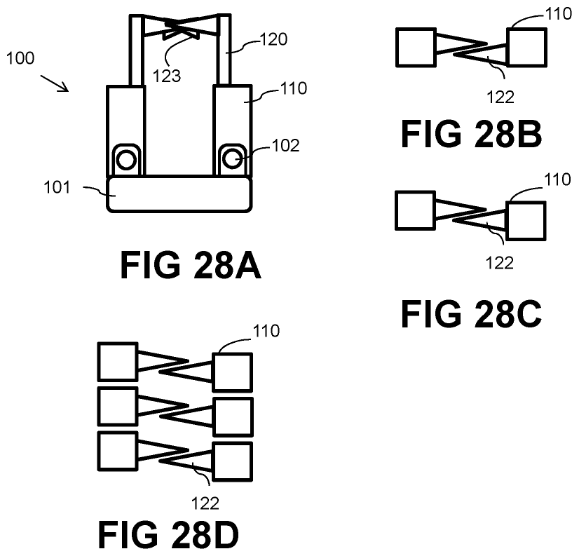

Referring to FIGS. 28A and 28B, such asymmetrical arrangement of the arms (or at least of the penetration means 122) may also be provided in a direction other than the axial. In this example, a relative displacement between the opposite penetration means is provided in a plane perpendicular to the axial direction (obviously, axial and non-axial offsets may also be combined). This may be achieved by asymmetric arrangement of the penetration means relative to its host elongated arm element (cf. FIG. 28B) or by asymmetric arrangement of the arms as such (cf. FIG. 28C). It will be understood that substantially the same effect can also be obtained by orienting (angulating) the penetration means off-axis with respect to the central axis instead of offsetting their position, e.g. such that the “left” arm is tilted slightly toward the “top” and the “right” arm slightly toward the “bottom” in the overhead view (e.g. similar to FIG. 28B; left, right, top and bottom refer to the relative frame of reference of such overhead view, for the sake of simplicity). Likewise, a rotation instead of displacement may be used to “fit” the penetration means together in the deployment configuration in the axial displacement example of FIG. 27A-B (which would result in the penetration means not being perfectly perpendicular to the elongated arm portion, but e.g. varying in pairs around the straight angle average).

While the approach shown in FIG. 28A-C may not easily generalize to more than two arms, it will be understood that the axial symmetry used throughout the present application is not necessarily limitative. For example, the arms may be arranged to form a clamp-like structure, as illustrated in FIG. 28D.

Furthermore, FIG. 28D shows an arrangement of a plurality (e.g. more than two) of arms using the same principle, in accordance with embodiments of the invention, whereby a claw-like structure is obtained, e.g. by positioning pairs of opposite arms (with respective penetration means) substantially parallel to each other with the penetration means on one side offset to fold in between the opposite penetration means on the other side.

FIG. 25 shows a flow-chart of a method 2500 of closing, or at least reducing the size of an opening 1261 of a human or animal body wall 1260. FIG. 12A to FIG. 17B illustrate corresponding situations. The method comprises the following steps:
a) providing 2501 an assembly 1250 (see FIG. 12A to FIG. 12D) comprising a biomedical device 1200 connected to a driver 1231, 1232. The biomedical device comprises a central component (e.g. a base), and a plurality of arms with penetration means. The arms may be connected to the central component by means of hinges. The biomedical device is configured in its delivery configuration;
b) inserting 2504 the biomedical device 1300 in the opening 1361 (see FIG. 13A to FIG. 13D);
c) rotating (or spreading) 2505 the arms 1420 of the biomedical device 1400 relative to its central axis A, thereby bringing the biomedical device in its deployed configuration (see FIG. 14A to FIG. 14D);
d) moving 2506 the biomedical device 1500 towards the opening, such that the penetration means of the biomedical device penetrates tissue surrounding the opening (see FIG. 15A and FIG. 15B);
e) moving 2508 at least some or all of the arms 1620 towards each other, e.g. by pulling threads or sutures 1634 connected to or looped through the arms (see FIG. 16B), thereby reducing or closing the opening, and bringing the biomedical device 1600 in its retracted configuration (see FIG. 17A and FIG. 17B);
f) disconnecting 2509 the central component (e.g. the base 1401) from the driver, e.g. by cutting some of the threads 1433 or sutures (see FIG. 14E).

The method may further comprise step g) of placing 2510 a clip (not shown) on some of the threads or sutures 1634, or making a knot with the threads or sutures. This step may be omitted if the arms are configured to move inside guiding elements, and in case clicking means or snapping means are provided preventing the arms from moving outwards again;

The method may further comprise step h) of stitching 2511 the tissue on the outside of the body.

Step a) may comprise: providing a biomedical device in the delivery configuration; and connecting the biomedical device to a driver, e.g. as illustrated in FIG. 2C to FIG. 2G.

The method may also comprise a further step of pulling 2507 the outer tube of the driver out of the opening. This step may be performed after step d) and before step e).

### REFERENCE SIGNS

The following reference signals (module 100) are used:
00 biomedical device
01 central component (e.g. base)
02 connection means (e.g. hinge, living hinge)
03 elongated groove or slit
04 holes in the base for connecting a driver
05 mounting opening (for connecting a guiding element to the base)
10 guiding element
11 clamping element
12 mounting opening of guiding element
20 arm
21 elongated portion of arm
22 penetration means (e.g. hook)
23 anchoring means (e.g. barb)
30 driver
31 first or inner tube of the driver
32 second or outer tube of the driver
33 first threads or sutures (for connecting a base to driver)
34 second threads or sutures (for pulling the arms)
35 additional tubular element
40 subassembly (arm+guiding element)
50 assembly (device+driver)
60 body wall
61 opening in body wall

1. A biomedical device (100; 2200) for closing an opening (1261) of a human or animal body wall (1260), the device comprising:
a central component (101; 2201) having a central axis (A), and
a plurality of arms (120; 2220) movably connected to the central component so as to transform the device between different configurations, said configurations comprising a delivery configuration, a deployed configuration and a retracted configuration;
wherein:
said arms (120; 2220) comprise an elongated arm portion (121), and penetration means (122; 2222) for penetrating said body wall;
the elongated arm portions (121) form an angle (L) smaller than 20° relative to the central axis (A) when the device is in said delivery configuration;
the elongated arm portions (121) are oriented substantially perpendicular to the central axis (A), and a tip of the penetration means (122) is located at a first distance (R1) from the central axis (A), when the device is in said deployed configuration;
the elongated arm portions (131) are oriented substantially perpendicular to the central axis (A), and the tip of the penetration means (122) is located at a second distance (R2) from the central axis (A), smaller than the first distance (R1), when the device is in said retracted configuration,
and wherein the penetration means (122) comprises a barb (123) for anchoring the penetration means in said body wall.

2. A biomedical device according to any of the previous claims,
wherein the biomedical device further comprises a plurality of guiding elements hingedly connected to the central component, configured for receiving respective elongated arm portions;
and wherein each arm is slidingly connected to a respective guiding element.

3. A biomedical device (2200) according to claim 2,
wherein the guiding elements have a shape configured for contacting each other in an edge-to-edge manner to form a smooth surface, when the device is in the deployed or retracted configuration.

4. A biomedical device (2100) according to any of the previous claims,
wherein the central component further comprises a plurality of clamping means, configured for receiving a respective arm between them;
and wherein the arms further comprise an elongated slot or a trench or a groove for engaging with the clamping means.

5. A biomedical device according to any of the previous claims,
wherein the penetration means (122) have an overall tapering shape or an overall pointed shape.

6. A biomedical device according to claim 5,
wherein said penetration means (122) has an overall pointed shape and said barb (123) has a pointed shape, smaller than said overall pointed shape of the penetration means, in which said pointed shape of the barb is oriented in a direction substantially opposite to the direction of said overall pointed shape of the penetration means.

7. A biomedical device according to any of the previous claims,
wherein said penetration means (122) is oriented substantially perpendicular to the elongated arm portion (121), such that the elongated arm portion and the penetration means together form an L-shape or T-shape.

8. A biomedical device according to any of the claims 1 to 6,
wherein said penetration means (122) and elongated arm portion (121) are curved so as, together, form an arc or C-shape.

9. A biomedical device according to any of the previous claims,
wherein the central component is a base having a first side with means for connecting the arms;
and having a second side with a smooth surface.

10. A biomedical device according to any of the previous claims,
wherein the central component has a rotational symmetrical shape about the central axis (A) of an order in the range from 2 to 8;
and/or wherein a projection of the central component is a substantially circular shape, or a substantially elliptical shape, or a substantially polygonal shape;
and/or wherein a projection of the biomedical device, when in its deployed configuration or its retracted configuration, has a rotational symmetrical shape of an order in the range from 2 to 8.

11. A biomedical device according to any of the previous claims,
wherein the biomedical device is made of a biocompatible material,
or wherein the biomedical device is made of a bioabsorbable material.

12. A biomedical device according to any of the previous claims,
wherein the central component has an interface for allowing a temporal connection to an external device (232) for allowing positioning and/or reconfiguration of the biomedical device.

13. A method of assembling a biomedical device according to any of the previous claims, comprising the steps of:
a) providing said central component (101);
b) providing said at least two elongated arms, and connecting them to the central component via said connection means.

14. A method according to claim 13, further comprising one or more of the following steps:
providing a first tube (231), and releasably connecting the first tube and the central component;
providing a second tube (232), and rotating the arms so as to align them in the first direction, and inserting at least the arms and optionally also the central component inside the second tube.

15. A method of closing an opening (1261) of a human or animal body wall using a biomedical device (100; 2200) according to any of the claims 1 to 12, comprising the steps of:
a) providing (2501) an assembly comprising a biomedical device connected to a driver, the biomedical device being configured in its delivery configuration;
b) inserting (2504) the biomedical device (100; 2200) in the opening (1261);
c) rotating (2505) the arms (120; 2220) of the biomedical device relative to its central axis (A), thereby bringing the biomedical device in its deployed configuration;
d) moving (2506) the biomedical device towards the opening, such that the penetration means of the biomedical device penetrates tissue surrounding the opening;
e) moving (2508) at least some of the arms towards each other, thereby reducing or closing the opening, and bringing the biomedical device in its retracted configuration.
